(19) United States
(12) Patent Application Publication
(10) Pub. No.: US 2010/0286070 A1
(43) Pub. Date: Nov. 11, 2010

(54) AFFINITY TAG

(76) Inventors: Gert Verheyden, Bavegem (BE); Fons Bosman, Opwijk (BE)

Correspondence Address: NIXON & VANDERHYE, PC, 901 NORTH GLEBE ROAD, 11TH FLOOR, ARLINGTON, VA, 22203, US

(21) Appl. No.: 12/733,591

(22) PCT Filed: Sep. 15, 2008
(86) PCT No.: PCT/EP2008/062250

§ 371 Date: Jun. 30, 2010

Related U.S. Application Data
(60) Provisional application No. 60/960,074, filed on Sep. 14, 2007.

(30) Foreign Application Priority Data
Sep. 14, 2007 (EP) 07116458.6

(51) Int. Cl. A61K 38/16; C07K 7/06; C07K 7/08; C07K 14/00; C07H 21/04; C12N 15/63; C07K 1/22; C07K 16/00; C12N 1/21; A61K 38/08; A61K 38/10; C12N 1/19; C12N 5/10; A61P 31/00; G01N 33/53

(52) U.S. Cl. 514/21.3; 530/328; 530/326; 530/350; 536/23.1; 435/320.1; 530/344; 530/327; 530/387.1; 435/252.3; 530/329; 530/325; 530/324; 514/21.7; 514/21.6; 514/21.5; 514/21.4; 435/254.2; 435/419; 435/325; 435/7.92

(57) ABSTRACT

The present invention relates to an affinity tag especially useful for human applications. The invention further includes methods for preparing fusion molecules, as well as compositions and reaction mixtures which contain said fusion molecules, nucleic acid molecules which encode these fusion molecules and recombinant host cells which contain these nucleic acid molecules.

### FIELD OF THE INVENTION

The present invention relates to an affinity tag particularly useful for human applications. The invention further includes fusion molecules, methods for preparing fusion molecules, as well as compositions containing said fusion molecules, nucleic acid molecules encoding these fusion molecules and recombinant host cells which contain these nucleic acid molecules.

### BACKGROUND OF THE INVENTION

Recombinant DNA technology has enabled the production of desired polypeptides in host cells. Such host-produced polypeptides typically are separated from host cell proteins prior to use. Affinity chromatography is often the preferred method for protein purification and can be used to purify proteins from complex mixtures with high yield. Affinity chromatography is based on the ability of proteins to bind non-covalently but specifically to an immobilized ligand for the desired protein, e.g. an antibody for a protein antigen. When the specific peptide has affinity to metal ions, isolation of the fusion protein can be done using metal affinity chromatography.

Immobilized Metal Ion Affinity Chromatography (IMAC) is one of the most frequently used techniques for purification of proteins. The technique is based on the natural ability of some proteins to bind to transition metals (Porath J. et al., 1975; Gaberc-Porekar and Menart, 2001). The affinity of proteins for transition metals derives from the properties of amino acids within the protein's primary structure which interact with and bind to metal ions. However, the occurrence of such metal/protein binding is random, due to the fact that not all proteins possess such metal binding amino acid sequences. Moreover, the strength of the bond to the metal ion varies unpredictably with the particular protein. Furthermore, if two or more protein molecules in a given mixture possess such metal binding sequences, the usefulness of the technique as a purification method is diminished since both will bind to the immobilized metal ion.

These shortcomings of the IMAC technique were solved by the advent of the CP-IMAC technique which provided a predictable and specific method for the purification of proteins by exploiting this natural protein-metal binding phenomenon. The term “CP-IMAC” reflects the use of “chelating peptides” or “tags” to specifically bind immobilized metal ions and purify proteins which contain such chelating peptides via IMAC principles. Chelating peptides or tags are short amino acid sequences which are specifically designed to interact with and bind to metal ions (Smith M C et al., 1988).

One of the most commonly used tags for affinity purification of proteins is the hexahistidine or (His)6 tag. However, only moderate purity from Escherichia coli extracts and relatively poor purification from yeast, Drosophila, and HeLa extracts are retrieved using a (His)6 tag (Lichty J J et al., 2005). E. coli SlyD, a prolyl isomerase, specifically binds divalent metal ions, which can result in significant contamination of IMAC preparations of heterologously expressed (His)6-tagged proteins. Moreover, clinical use of proteins carrying a (His)6 tag is very limited as numerous human proteins share motives of (His)6 and the (His)6 sequence is a known B-cell epitope (Kim et al., 2001). In order to reduce or avoid a safety risk, it is recommended to remove the his-tag before the molecule can be used on or in the human body. This implicates however a labor intensive and costly production process.

Although other tags exist such as glutathion-s-transferase (GST), strep II (STR), FLAG peptide, heavy chain of protein C (HPC), maltose binding protein (MBP), covalent yet dissociable (CYD), and calmodulin binding protein (CBP), these have usually even more limitations to their use than the (His)6 tag and raise additional concerns for human applications.

The current invention identified novel tags allowing purification based on affinity chromatography, with excellent purification properties, and that no longer share extensive homology with the human genome. Therefore, the tags of the present invention are especially useful for large-scale purification at low cost with high product recoveries. Furthermore, removal of the tag for production of clinical-grade proteins is not needed, thanks to their low human homology characteristics.

### SUMMARY OF THE INVENTION

In a first embodiment, the affinity tag of the invention consists of a sequence of 7 to 50 amino acids with the following characteristics:
comprising at least 6 Histidine residues whereby each His residue is followed by another His residue or by 1 to 4 non-His amino acids,
wherein at most 4 consecutive amino acids of the tag sequence are identical to a human protein amino acid sequence, and
wherein any window of 6 consecutive amino acids of the tag sequence can comprise up to 5 amino acids identical to a human protein sequence but with the provision that the remaining amino acid in the window is not similar to a human protein amino acid in said window.

More specific, the affinity tag comprises the sequence H(X₁)(X₂)(X₃)(X₄)H(X₅)(X₆)(X₇)(X₈)H(X₉)(X₁₀)(X₁₁)(X₁₂)H(X₁₃)(X₁₄)(X₁₅)(X₁₆)H(X₁₇)(X₁₈)(X₁₉) (_{X20})H (SEQ ID NO 36), wherein X₁₋₂₀ are, independently from each other, either optional, or if present selected from any non-His amino acid. In a preferred embodiment, X₁₋₂₀ are independently from each other selected from the group of amino acids consisting of M, W, N and F. The indication of X between brackets —(X)— means optional.

In a further embodiment of the invention the affinity tag comprises the sequence (H)HH—X₁—X₂—(H)HH (SEQ ID NO 37), whereby X₁₋₂ are independently from each other selected from the group consisting of N, M, F and W. Even more particular, the affinity tag comprises the sequence (H)HH—X₁—X₂—(H)HH—X₃—X₄—(H)HH—X₅—X₆—(H)HH (SEQ ID NO 38), whereby X₁₋₆ are independently from each other selected from the group consisting of N, M, F and W. The indication of H between brackets “(H)” means optional.

In a specific embodiment, the affinity tag of the present invention comprises a sequence selected from the group consisting of the sequences represented by SEQ ID NO 1 to SEQ ID NO 35. Even more specific, the affinity tag comprises the sequence selected from the group consisting of

| | |
|---|---|
| HHHWWHHH; | (SEQ ID NO 1) |
| HHHWWHHHWWHHH; | (SEQ ID NO 17) |
| HHHWWHHHWWHHHWWHHH; | (SEQ ID NO 33) |
| HHMWHHHMWHHH; | (SEQ ID NO 13) |
| HHMWHHHMWHHHMWHHH; | (SEQ ID NO 31) |
| HHHMFHHNWHH; | (SEQ ID NO 12) |
| HHHMFHHHWWHHH; | (SEQ ID NO 22) |
| HHHWWHHHMWHHH; | (SEQ ID NO 23) |
| and | |
| HHHMFHHHWWHHHMWHHH. | (SEQ ID NO 32) |

In a particular embodiment, the tag sequence comprises at least 8 Histidine (H) residues. Even more particular, the tag is a metal affinity tag.

In another aspect, the invention encompasses a fusion molecule which is a molecule linked to the tag as described herein. The tag is linked directly to the molecule or via a linker In a preferred embodiment, the linker is a peptide sequence of 1 to 30 amino acids long. More specific, the molecule linked to the tag is a protein or a fragment thereof Preferably, said protein or fragment thereof is immunogenic.

The present invention further relates to an isolated nucleic acid fragment coding for the affinity tag as described herein, an isolated nucleic acid comprising said nucleic acid fragment, and an isolated nucleic acid coding for the fusion molecule as described herein. The invention also encompasses a vector comprising said nucleic acid(s) and a host cell comprising said vector or nucleic acid(s).

In a further embodiment, the present invention relates to the use of the affinity tag, or the nucleic acid encoding it, for several and diverse applications. The tag is especially useful for the purification or immobilization of a molecule. Accordingly, the present invention is furthermore directed to a method for purifying a fusion molecule comprising the steps of:

(a) applying a solution containing a fusion molecule described herein to a solid support possessing an immobilized affinity ligand,

(b) forming a complex between said immobilized affinity ligand and said molecule,

(c) removing weakly bounded molecules, and

(d) eluting the bound molecule.

Optionally, the affinity tag is removed in a subsequent step (e).

The invention also relates to a method for immobilizing a fusion molecule comprising the steps of:

(a) applying a solution containing a fusion molecule of the invention to a solid support possessing an immobilized affinity ligand,

(b) forming a complex between said immobilized affinity ligand and said molecule, and

(c) removing weakly bounded molecules.

A further method of the invention is directed to the detection of a molecule in a sample by using the affinity tag of the invention, or the nucleic acid encoding it as a marker.

In a preferred embodiment, the affinity ligand is a metal ion charged IMAC ligand, an antibody, an antibody fragment, a small molecule or a synthetic affinity ligand.

A further embodiment of the invention relates to a composition, more particular a pharmaceutical composition, comprising the fusion molecule as described herein. Optionally, the composition further comprises at least one of a pharmaceutically acceptable excipient.

The invention is furthermore directed to the fusion molecule, or composition comprising it, for use as a medicament. More specific, the molecule linked to the tag is an immunogenic compound. The invention also relates to a method for preparing the fusion molecule and to an antibody specifically binding the affinity tag or fusion molecule of the invention.

### FIGURE LEGENDS

FIG. 1: Recoveries of peptides after Ni²⁺-IMAC.

FIG. 2: A. Amino acid sequence of the HBV polyepitope protein.

B. Amino acid sequence of the HCV polyepitope protein.

FIG. 3: Nucleic acid sequence of the HBV polyepitope protein with linker and tag LHH-03.

FIG. 4: Nucleic acid sequence of the HBV polyepitope protein with linker and tag LHH-07.

FIG. 5: Nucleic acid sequence of the HBV polyepitope protein with linker and tag LHH-08.

FIG. 6: Nucleic acid sequence of the HBV polyepitope protein with linker and tag LHH-09.

FIG. 7: Nucleic acid sequence of the HBV polyepitope protein with linker and tag LHH-11.

FIG. 8: Nucleic acid sequence of the HCV polyepitope protein with linker and tag LHH-08.

FIG. 9: Nucleic acid sequence of the HCV polyepitope protein with linker and tag LHH-11.

FIG. 10: A. Recoveries obtained after IMAC of HBV fusion constructs, expressed in E. coli SG40440 (pcI857) strains.

B. Recoveries obtained after IMAC of HBV fusion constructs, expressed in E. coli BL21 (pAcI) strains.

FIG. 11: SDS-PAGE analysis and subsequent silver staining of purified HBV fusion constructs, expressed in E. coli SG40440 (pcI857) and E. coli BL21 (pAcI) strains.

FIG. 12: Recoveries obtained after IMAC of HCV fusion constructs, expressed in E. coli SG40440 (pcI857) strains.

FIG. 13: A. SDS-PAGE and Coomassie staining on IMAC samples of HCV LHH-08 fusion construct, expressed in E. coli SG40440 (pcI857) strain.

B. SDS-PAGE and Coomassie staining on IMAC samples of HCV LHH-11 fusion construct, expressed in E. coli SG40440 (pcI857) strain.

FIG. 14: SDS-PAGE and Silver staining on IMAC samples of the HCV fusion construct, expressed in E. coli SG40440 (pcI857) strain.

FIG. 15: Restriction map of plasmid pAcI (ICCG1396).

FIG. 16: Nucleic acid sequence of the plasmid pAcI (1-4947 bps).

FIG. 17: Restriction map of the plasmid pcI857 (ICCG167).

FIG. 18: Nucleic acid sequence of the plasmid pcI857 (1-4182 bps).

FIG. 19: A. Set-up Peptide coating ELISA

B. Set-up HCV or HBV poly-epitope protein coating ELISA

### DETAILED DESCRIPTION OF THE INVENTION

The present invention has identified new affinity tags with improved properties that are especially useful for human applications.

Candidate affinity tags were screened by NCBI Blast searching (National Center for Biotechnology Information, NLM/NIH) against the human genome. The following cut-off was used and defines the term “low human homology” (LHH):
no human protein should have 5 or more consecutive amino acids identical to 5 or more consecutive amino acids of the tag; and
no human protein should share a window of 6 amino acids in which 5 are identical and the sixth amino acid is considered to be a conservative substitution compared to the tag.

The term “window” refers to a series of consecutive amino acids. For example, a window of 6 amino acids is a series of 6 consecutive amino acids.

The screening finally resulted in a pool of peptides with low human homology (Example 1).

From this pool, a random subset of candidate tags were produced as a biotinylated peptide and their binding to Ni-IMAC was assessed. As shown in the Examples section, all the tags demonstrated to be efficient for use in purification. Preferably and in order to obtain good binding properties, the affinity tag should comprise at least 6 Histidine residues, i.e. 6, 7, 8, 9, 10, 11, 12, 13, 14, 15, 16, 17, 18, 19, 20, 25, or more His residues, possibly up till 50 or 100 His residues. If higher purification performance than obtained with the standard His6 tag is desired (e.g. with respect to SlyD removal), the affinity tag preferentially comprises at least 8 His residues.

In a preferred embodiment, the His residues are organized as a single residue (H), doublets (HH), triplets (HHH) and/or quartets (HHHH). In a more preferred embodiment, the His residues are organized into doublets (HH) and/or triplets (HHH). Furthermore, each His residue in the tag sequence is followed by another His residue or by 1 to 4 (i.e. 1, 2, 3, or 4) non-His amino acids. The human homology search furthermore showed that said non-His amino acids are preferably selected from the group consisting of N (Asn, Asparagine), M (Met, Methionine), F (Phe, Phenylalanine) and W (Trp, Tryptophan).

In a first embodiment, the present invention relates to an isolated affinity tag consisting of a sequence of 7 to 50 amino acids with the following characteristics:
comprising at least 6 Histidine residues whereby each His residue is followed by another His residue or by 1 to 4 non-His amino acids,
wherein at most 4 consecutive amino acids of the tag sequence are identical to a human protein amino acid sequence, and
wherein any window of 6 consecutive amino acids of the tag sequence can comprise up to 5 amino acids identical to a human protein sequence but with the provision that the remaining amino acid in the window is not similar to a human protein amino acid in said window.

The term “similar” refers to a conservative substitution of one amino acid by another at a given position in an alignment. The limits of said term are set by the NCBI blast searching program (blastp). If the aligned residues have similar physico-chemical properties, the substitution is said to be “conservative”. The search settings for the blast program in the present invention are given in Example 1. An amino acid “dissimilar” to another, means that the amino acid cannot be considered as a conservative substitution for the other.

In a particular embodiment, the affinity tag consists of at least 7, 8, 9, 10, 11, 12, 13, 14, 15, 16, 17, 18, 19, 20, 21, 22, 23, 24, 25 or more amino acids. The upper limit of the length of the tag is less pertinent and will depend on e.g. chromatographic elution behavior preferred and can vary form 8, 10, 15, 20, 25, 30, 35, 40, 45, 50, even up to100 amino acids, and including every value in between.

As used herein, a “non-His amino acid” is any amino acid other than L- or D-Histidine, and is preferably selected from the following table (Table 1), including isomers thereof, rare amino acids and synthetically modified residues:

TABLE 1

| Amino Acid | Short | Abbrev. |
|---|---|---|
| Alanine | A | Ala |
| Cysteine | C | Cys |
| Aspartic acid | D | Asp |
| Glutamic acid | E | Glu |
| Phenylalanine | F | Phe |
| Glycine | G | Gly |
| Isoleucine | I | Ile |
| Lysine | K | Lys |
| Leucine | L | Leu |
| Methionine | M | Met |
| Asparagine | N | Asn |
| Proline | P | Pro |
| Glutamine | Q | Gln |
| Arginine | R | Arg |
| Serine | S | Ser |
| Threonine | T | Thr |
| Valine | V | Val |
| Tryptophan | W | Trp |
| Tyrosine | Y | Tyr |

In a further embodiment, the affinity tag comprises or consists of the sequence: H(X₁)(X₂)(X₃)(X₄)H(X₅)(X₆)(X₇)(X₈)H(X₉)(X₁₀)(X₁₁)(X₁₂)H(X₁₃)(X₁₄)(X₁₅)(X₁₆)H(X₁₇)(X₁₈)(X₁₉) (_{X20})H (SEQ ID NO 36), wherein X₁₋₂₀ are, independently from each other either optional, or, if present selected from any non-His amino acid.

In a preferred embodiment, one or more X-residues are independently from each other selected from the group of amino acids consisting of N, M, F and W. The indication of X between brackets —(X)— means optional, i.e. present or not.

In a further embodiment, the affinity tag comprises or consists of the sequence (H)HH—X₁—X₂—(H)HH (SEQ ID NO 37), whereby X₁₋₂ are independently from each other selected from the group consisting of N, M, F and W. More specific, X₁₋₂ is selected from the group consisting of M, F and W. The indication of H between brackets “(H)” means optional, i.e. present or not.

In a preferred embodiment, at least one X is W. It is clear that said sequence, or parts thereof, can be combined and/or repeated two, three, four, five or more times. In a more particular embodiment, the current invention is directed to an affinity tag comprising or consisting of the sequence (H)HH—X₁—X₂—(H)HH—X₃—X₄—(H)HH—X₅—X₆—(H)HH (SEQ ID NO 38), wherein X₁₋₆ are independently from each other selected from the group consisting of N, M, F and W. More specific, X₁₋₆ is selected from the group consisting of M, F and W; or, X₁₋₆ is selected from the group consisting of M, F and W and at least one X is W; or, X₁₋₆ is selected from the group consisting of M, F and W and at least two X residues are W; or, X₁₋₆ is selected from the group consisting of M, F and W and at least three X residues are W; or, X₁₋₆ is W. The indication of H between brackets “(H)” means optional, i.e. present or not.

In an even more particular embodiment, the affinity tag comprises or consists of an amino acid sequence selected from the group consisting of the sequences represented by SEQ ID NO 1 to SEQ ID NO 35. More specific, the affinity tag comprises or consists of a sequence selected from the group consisting of

HHHWWHHH (SEQ ID NO 1); HHHWWHHHWWHHH (SEQ ID NO 17); HHHWWHHHWWHHHWWHHH (SEQ ID NO 33); HHMWHHHMWHHH (SEQ ID NO 13); HHMWHHHMWHHHMWHHH (SEQ ID NO 31); HHHMFHHNWHH (SEQ ID NO 12); HHHMFHHHWWHHH (SEQ ID NO 22); HHHWWHHHMWHHH (SEQ ID NO 23); and HHHMFHHHWWHHHMWHHH (SEQ ID NO 32). Even more specific, the affinity tag comprises or consists of the following sequence: HHHMFHHNWHH (SEQ ID NO 12) or HHHMFHHHWWHHHMWHHH (SEQ ID NO 32).

As used herein, the term “affinity tag” refers to a peptide enabling a specific interaction with a specific ligand.

In one embodiment, the affinity tag is linked to a molecule said combination being referred to herein as a “fusion molecule” or “fusion construct”. Accordingly, the present invention relates to a fusion molecule comprising the affinity tag as described herein. The affinity tag can be linked directly or indirectly to said molecule. The tag can be linked to any site of the molecule, e.g. to or near the end or terminus of the molecule, to one or more internal sites, attached to a side chain, or to the amino-terminal amino acid (N-terminal) or to the carboxy-terminal amino acid (C-terminal). Also more than one affinity tag can be linked to the molecule.

In the case of indirect linking, a suitable linker sequence is inserted between the tag and the desired molecule. The affinity tag can then be removed chemically or enzymatically if a cleavage site is present in the linker sequence, using methods known in the art. Preferred linkers are peptides of 1 to 30 amino acids long and include, but are not limited to the peptides EEGEPK (Kjeldsen et al. in WO98/28429; SEQ ID NO 39) or EEAEPK (Kjeldsen et al. in WO97/22706; SEQ ID NO 40); the G4S immunosilent linker; a protease cleavage site such as Factor Xa cleavage site having the sequence IEGR (SEQ ID NO 41), the thrombin cleavage site having the sequence LVPR (SEQ ID NO 42) or the enteroskinase cleaving site having the sequence DDDDK (SEQ ID NO 43). Preferably, the amino acid linker sequence is selected so that the amino acid sequence obtained by said linker in combination with the neighboring molecule sequence and tag sequence fulfill the low human homology criterium as defined herein. Other suitable linkers are carbohydrates, PEG based linkers, and other available in the art. The term “molecule” as used herein refers to proteins, including antibodies and enzymes, peptides, nucleotides, lipids, carbohydrates, drugs and cofactors, or combinations thereof. The molecule may be of varying length, size or molecular weight, and can have any activity known and desired by the skilled person. The molecule as described herein is “isolated”. The term “isolated” refers to material that is substantially free from components that normally accompany it as found in its naturally occurring environment.

In a specific embodiment, the molecule is immunogenic. The term “immunogenic” or “immunogenicity” or “immunoreactive” as used herein is the ability to evoke an immune response, i.e. a humoral and/or cellular response. The term “humoral immune response” refers to an immune response mediated by antibody molecules, while a “cellular immune response” is one mediated by T-lymphocytes and/or other white blood cells.

Immunogenicity can be manifested in several different ways. Immunogenicity corresponds to whether an immune response is elicited at all, and to the vigor of any particular response, as well as to the extent of a population in which a response is elicited. Preferred immunogenic molecules or compounds may be determined by a variety of methods. For example, identification of immunogenic portions of a protein may be predicted based upon amino acid sequence. Briefly, various computer programs which are known to those of ordinary skill in the art may be utilized to predict CTL and HTL epitopes. Other assays, however, may also be utilized, including, for example, ELISA which detects the presence of antibodies against the molecule, as well as assays which test for CTL and/or HTL epitopes, such as ELISPOT and proliferation assays.

In a particular embodiment, the molecule is a protein. More particular, the affinity tag of the invention is coupled to a protein, or a fragment thereof, said combination also being referred to as a fusion protein. In fact and as used herein, a “fusion protein” refers to a polypeptide which comprises the amalgamation of two amino acid sequences derived from heterogeneous sources. The protein can have any activity known and desired by the skilled person, e.g. immunogenic activity, enzymatic activity, or binding activity. Specific proteins, including fragments thereof, which can be linked to the affinity tag of this invention, include for example enzymes, cytokines, intracellular signaling peptides, receptors, antibodies, vaccine components, and synthetic peptides. In a specific embodiment, the proteins are derived from bacteria or viruses, e.g. proteins derived from the Hepatitis B virus (HBV), such as but not limited to three HB “Surface” antigens (HBsAgs): an HBcore antigen (HBcAg), an HBe antigen (HBeAg), and an HBx antigen (HBxAg). Also presented by HBV are polymerase (“HBV pol”), open reading frame 5 (ORF 5), and ORF 6 antigens. Other possible polypeptides or proteins are derived from the Hepatitis C virus (HCV), e.g. UTR, Core, E1, E2, NS3, NS4 and NS5, or from the Human Papillomavirus (HPV), such as the L1, L2, E1, E2, E4, E5, E6 and E7 protein.

As will be evident to one of ordinary skill in the art, various immunogenic portions or fragments of the herein described proteins may be combined. Said immunogenic portion(s) or fragment(s) may be of varying length, although it is generally preferred that these are at least 7 amino acids long, and up to the length of the entire protein.

In a preferred embodiment, the protein is a polyepitope construct. In a particular embodiment, the affinity tag of the present invention is linked to a polyepitope construct. The current invention thus also relates to a polyepitope construct coupled to the affinity tag described herein. The term “polyepitope” refers to the inclusion of more than two epitopes. The term “construct” as used herein generally denotes a composition that does not occur in nature. As such, the polyepitope construct of the present invention is not a wild-type full-length protein but is a chimeric protein containing isolated epitopes from at least one protein, not necessarily in the same sequential order as in nature. With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) molecules. With regard to a particular nucleic acid sequence, a “nucleic acid epitope” is a set of nucleic acids that encode for a particular amino acid sequence that forms an epitope. Specific characteristics of a polyepitope construct and methods for designing and producing it are given e.g. in WO01/47541, WO04/031210 and WO05/089164.

The epitopes can be derived from any desired protein of interest, e.g. a viral protein, a tumor protein or any pathogen. Multiple HLA class I or class II epitopes present in a polyepitope construct can be derived from the same antigen, or from different antigens. For example, a polyepitope construct can contain one or more HLA binding epitopes than can be derived from two different antigens of the same virus, or from two different antigens of different viruses.

The preparation of the fusion molecules of this invention can be carried out using standard recombinant DNA methods.

In a specific embodiment, the present invention relates to an isolated nucleic acid fragment that encodes the affinity tag described herein, an isolated nucleic acid comprising said nucleic acid fragment, an isolated nucleic acid encoding the fusion molecule of the invention, as well as to a composition comprising such a nucleic acid molecule. Nucleic acid molecules may be DNA, RNA, or combinations thereof. The nucleic acid segments that encode the molecule and the affinity tag may be contiguous, such that in the transcription and/or translation products of the coding segments, the segments are juxtaposed. In some embodiments, the coding sequences of the tag of the invention and a molecule may be separated by a linker encoding nucleic sequence, or by one or more sequences that are non-coding. Thus, the present invention encompasses nucleic acid molecules containing one or more intervening sequences (e.g.introns) that may be transcribed from a DNA molecule into an RNA molecule and subsequently removed (e.g. by splicing) prior to translation of the RNA molecule into protein. Nucleic acid molecules of the invention may be synthesized in vitro, in vivo, or by the action of cell-free transcription. Preferably, a nucleotide sequence coding for the affinity tag is first synthesized and then linked to a nucleotide sequence coding for the desired molecule or protein.

The thus-obtained hybrid gene can be incorporated into an expression or cloning vector using standard methods. Vectors according to this aspect of the invention can be double-stranded or single-stranded and may be DNA, RNA, or DNA/RNA hybrid molecules, in any conformation including but not limited to linear, circular, coiled, supercoiled, torsional, nicked and the like. These vectors of the invention include but are not limited to plasmid vectors and viral vectors, such as a bacteriophage, baculovirus, retrovirus, lentivirus, adenovirus, vaccinia virus, semliki forest virus and adeno-associated virus vectors, all of which are well-known and can be purchased from commercial sources. Any vector may be used to construct the fusion molecules used in the methods of the invention. In particular, vectors known in the art and those commercially available (and variants or derivatives thereof) may in accordance with the invention be engineered to include one or more recombination sites for use in the methods of the invention. General classes of vectors of particular interest include prokaryotic and/or eukaryotic cloning vectors, expression vectors, fusion vectors, two-hybrid or reverse two- hybrid vectors, shuttle vectors for use in different hosts, mutagenesis vectors, transcription vectors, vectors for receiving large inserts and the like. Other vectors of interest include viral origin vectors (M13 vectors, bacterial phage 8 vectors, adenovirus vectors, and retrovirus vectors), and high, low and adjustable copy number vectors. Most of the requisite methodology can be found in Ausubel et al., 2007.

DNA constructs prepared for introduction into a prokaryotic or eukaryotic host will typically comprise a replication system recognized by the host, including the intended DNA fragment encoding the fusion molecule of the present invention, and will preferably also include transcription and translational initiation regulatory sequences operably linked to the molecule-encoding segment. Expression systems may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included, where appropriate, from secreted polypeptides of the same or related species, which allow the protein to cross and/or lodge in cell membranes, or be secreted from the cell.

An appropriate promoter and other necessary vector sequences will be selected so as to be functional in the host. Examples of workable combinations of cell lines and expression vectors are described in Sambrook et al. (1989), Ausubel et al. (Eds.) (2007), and Metzger et al. (1988). Many useful vectors for expression in bacteria, yeast, fungal, mammalian, insect, plant or other cells are well known in the art. In addition, the construct may be joined to an amplifiable gene (e.g., DHFR) so that multiple copies of the gene may be made. For appropriate enhancer and other expression control sequences, see also Enhancers and Eukaryotic Gene Expression (1983) Cold Spring Harbor Press, N.Y. While such expression vectors may replicate autonomously, they may less preferably replicate by being inserted into the genome of the host cell.

Expression and cloning vectors will likely contain a selectable marker, that is, a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that (a) confer resistance to antibiotics e.g., kanamycin, tetracycline, etc. or other toxic substances; (b) complement auxotrophic deficiencies; or (c) supply critical nutrients not available from complex media. The choice of the proper selectable marker will depend on the host cell; appropriate markers for different hosts are known in the art.

Recombinant host cells, in the present context, are those which have been genetically modified to contain an isolated DNA molecule of the instant invention. The DNA can be introduced by any means known to the art which are appropriate for the particular type of cell, including without limitation, transformation, lipofection, electroporation or viral mediated transduction. A DNA construct capable of enabling the expression of the fusion molecule of the invention can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989), Maniatis et al. (1982), Wu (ed.) (1993) and Ausubel et al. (1992).

In a further embodiment, the present invention encompasses host cells comprising one or more nucleic acid molecules of the invention (e.g. a nucleic acid molecule encoding one or more fusion molecules of the invention). Representative host cells that may be used with the invention include, but are not limited to, bacterial cells, yeast cells, plant cells and animal cells. Bacterial host cells suitable for use with the invention include Escherichia spp. cells (particularly E. coli cells and most particularly E. coli strains BL21 and SG4044), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aerugitiosa cells), and Salmonella spp. cells (particularly S. typhimurium and S. typhi cells). Animal host cells suitable for use with the invention include insect cells (most particularly Drosophila melanogaster cells, Spodoptera frugiperda Sf9 and Sf21 cells and Trichoplusa High-Five cells), nematode cells (particularly C. elegant cells), avian cells, amphibian cells (particularly Xenopus laevis cells), reptilian cells, and mammalian cells (most particularly derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2). Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively the host cells may also be transgenic animals.

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, transfection, and transformation. The nucleic acid molecules and/or vectors of the invention may be introduced alone or in conjunction with other nucleic acid molecules and/or vectors. Alternatively, the nucleic acid molecules and/or vectors of the invention may be introduced into host cells as a precipitate, such as a calcium phosphate precipitate, or in a complex with a lipid.

Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook J et al., pp. 16.30-16. 55 (1989), Watson J D et al., pp. 213-234 (1992), and Winnacker, E., (1987), which are illustrative of the many laboratory manuals that detail these techniques and which are incorporated by reference herein in their entireties for their relevant disclosures.

The affinity tags of the present invention may serve any purpose including but not limited to:
make a fusion molecule suitable for particular purification methods,
make a fusion molecule suitable for covalent or non-covalent immobilization,
enable one to identify whether a fusion molecule is present in a sample or composition,
tissue-specific localization of a fusion molecule,
(intra)cellular targeting,
labeling function, and
enabling drug delivery of e.g. oligonucleotides.

In a specific embodiment, the present invention relates to the purification of molecules comprising one or more affinity tags of the invention. The affinity tags allow molecules to be purified using generalized protocols in contrast to highly customized procedures associated with conventional chromatography. Moreover, use of these tags provides the superior advantages of traditional metal affinity tags (e.g. hexahistidine tag) compared to other affinity tags, namely suitability for use in large-scale purification at low cost, possible purification under denaturing concentrations of urea or guanidine-HCl, on-column refolding, high product recoveries, etc., and makes removal of the tag for production of clinical-grade proteins (to reduce risk to elicit an adverse immune response against the tag) superfluous, thanks to their low human homology characteristics.

Fusion molecules may be purified from the host cell or from the host cell culture medium into which they have been secreted. Typically, when purified from a host cell, the host cell is lysed using standard techniques (e.g., enzymatic digestion, sonication, French press, etc.) to form a lysate comprising the fusion molecule. Said fusion molecule may be purified from a lysate or from a host cell culture medium material by contacting the lysate or medium with a suitable chromatography medium under conditions suitable for binding of the fusion molecule to the chromatography medium. The lysate or culture medium may be contacted with a chromatography medium in either a batchwise technique (e.g. by mixing the chromatography medium with the lysate or culture medium) or column technique. The resin bound fusion molecule may be washed one or more times to remove any weakly bounded materials, i.e. materials that do not bind as tightly as the fusion molecule to the chromatography medium. The molecule may then be eluted from the medium by contacting the medium with a suitable elution buffer known to the skilled person, e.g. imidazole. The elution of the fusion molecule from the column can be carried out at a constant pH or with linear or discontinuously falling pH gradients. The optimal elution conditions depend on the amount and type of impurities which are present, the amount of material to be purified, the column dimensions, the chromatography resin used, etc. and are easily determined by routine experimentation on a case-by-case basis.

In a particular embodiment, the invention relates to a method of purifying a fusion molecule comprising the steps of:

(a) applying a solution containing a molecule linked to the affinity tag as described herein to a solid support possessing an immobilized affinity ligand,

(b) forming a complex between said immobilized affinity ligand and said molecule,

(c) removing weakly bounded molecules, and

(d) eluting the bound molecule.

As used herein, the “affinity ligand” binds to the tag of the present invention and can be any molecule and more particular a metal affinity ligand, an antibody, an antibody fragment, a small molecule or a synthetic affinity ligand.

As discussed herein, a fusion molecule may comprise a cleavage site for a protease, for example, located between the tag of the invention and a molecule of interest. After elution from the chromatography medium or while still bound to the medium, a fusion molecule of the invention may be contacted with a solution comprising a protease enzyme that cleaves at the cleavage site. In a specific embodiment, the purification method further comprises a step (e) wherein the affinity tag is removed.

The purification method may be any method known in the art. Suitable purification methods include but are not limited to affinity chromatography (e.g. Immobilized Metal Ion Affinity Chromatography (IMAC)), immunoaffinity chromatography, metal-affinity precipitation, immobilized-metal-ion-affinity electrophoresis and Immunoprecipitation.

In case of chromatography, the affinity column contains a solid support (e.g. resin) with one or more of the following: Fe, Co, Ni, Cu, Zn, or Al charged IMAC ligand, an antibody, an antibody fragment, a small molecule or a synthetic affinity ligand (e.g. aptamers or ligands derived using Versaffin™)

As is demonstrated herein, the use of the affinity tag of the present invention for purification results in highly purified proteins with a good yield.

The term “purity” or “purified” as applied to proteins herein implies that the desired protein preferably comprises at least 60%, more preferably at least 70%, more preferably at least about 80%, still more preferably at least about 90%, and most preferably at least about 95% of the total protein component.

The present invention furthermore encompasses a method for immobilizing a fusion molecule on a support comprising the steps of:

(a) applying a solution containing a molecule linked to the affinity tag as described herein to a solid support possessing an affinity ligand,

(b) forming a complex between said immobilized affinity ligand and said molecule, and

(c) removing weakly bounded molecules.

The method may be performed using immobilized elements and the immobilization may be carried out using a variety of immobilization means (e.g., columns, beads, adsorbents, nitrocellulose paper, etc.). The immobilization assay can be used to screen a sample for antibodies against the molecule linked to the tag. Furthermore, the tag of the invention can be part of a screening assay in order to screen large libraries of test compounds (e.g., drugs, new antimicrobials, etc.). The screening assay is preferably conducted in a microplate format. Any means or method of detection can be used. For example, the detection means might be a plate reader, a scintillation counter, a mass spectrometer or fluorometer.

The invention further relates to a method for identifying whether a molecule is present in a sample or composition. For example, the affinity tag of the present invention can be used in detection of a molecule via anti-tag antibodies in gel staining (SDS-PAGE). This can be useful in subcellular localization, ELISA, western blotting or other immuno-analytical methods.

Antibodies specifically binding the herein described tag are also part of the invention.

Antibodies may be polyclonal and/or monoclonal. They may be prepared against the entire affinity tag or against a fragment of the tag. As used herein, the term “antibody” (Ab) is meant to include whole antibodies, including single-chain whole antibodies, and antigen-binding fragments. In some embodiments, antigen-binding fragments may be mammalian antigen-binding antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain.

One or more of the affinity tags and/or fusion molecules of the invention may be used as immunogens to prepare polyclonal and/or monoclonal antibodies capable of binding the affinity tags and/or fusion molecules using techniques well known in the art (Harlow & Lane, 1988). In brief, antibodies are prepared by immunization of suitable subjects (e.g. mice, rats, rabbits, goats, etc.) with all or a part of the affinity tags and/or fusion molecules of the invention. If the affinity tag and/or fusion molecule, or a fragment thereof, is sufficiently immunogenic, it may be used to immunize the subject. If necessary or desired to increase immunogenicity, the affinity tag and/or fusion molecule, or fragment, may be conjugated to a suitable carrier molecule (e.g., BSA, KLH, and the like).

Monoclonal antibodies can be prepared from the immune cells of animals (e.g. mice, rats, etc.) immunized with all or a portion of one or more affinity tags and/or fusion molecules of the invention using conventional procedures, such as those described by Kohler and Milstein (1975). Thus, the present invention provides monoclonal antibodies specific to the affinity tag and/or fusion molecule of the invention, as well as cell lines producing such monoclonal antibodies. Antibodies of the invention may be prepared from any animal origin including birds and mammals.

Antibodies may be used for the detection of the affinity tag in an immunoassay, such as ELISA, Western blot, radioimmunoassay, enzyme immunoassay, and may be used in immunocytochemistry. In some embodiments, an anti-tag antibody may be in solution and the tag to be recognized may be in solution (e.g. an immunopreciptitation) or may be on or attached to a solid surface (e.g. a Western blot). In other embodiments, the antibody may be attached to a solid surface and the tag may be in solution (e.g. immunoaffinity chromatography or ELISA).

Antibodies to the tags and/or fusion molecules of the invention may be used to determine the presence, absence or amount of one or more molecules in a sample. The amount of specifically bound tag and/or fusion molecule may be determined using an antibody to which a marker is attached, such as a radioactive, a fluorescent, or an enzymatic label. Alternatively, a labeled secondary antibody (e.g. an antibody that recognizes the antibody that is specific to the polypeptide) may be used to detect a polypeptide-antibody complex between the specific antibody and the polypeptide.

The present invention furthermore relates to a composition comprising the fusion molecule as described herein. In a particular embodiment, the composition is a pharmaceutical composition. More specific, the composition furthermore comprises at least one of a pharmaceutically acceptable excipient, i.e. a carrier, adjuvant or vehicle, well known to the skilled person in the art. The terms “immunogenic composition” and “pharmaceutical composition” can be used interchangeably. More particularly, said immunogenic composition is a vaccine composition. Even more particularly, said vaccine composition is a therapeutic vaccine composition. Alternatively, said vaccine composition may also be a prophylactic vaccine composition. In a particular embodiment, the invention encompasses the fusion molecule as described herein for use as a medicament. In a preferred embodiment, the molecule linked to the tag is an immunogenic compound.

The affinity tag of the present invention allows immunogenic molecules to be purified using generalized protocols in contrast to highly customized procedures associated with conventional chromatography. Moreover, removal of the tag for production of clinical-grade molecules (to reduce risk to elicit an adverse immune response against the tag) is superfluous, thanks to the low human homology characteristics of the tag.

It is to be understood that although preferred embodiments, specific constructions and configurations, as well as materials, have been discussed herein for the methods and tools according to the present invention, various changes or modifications in form and detail may be made without departing from the scope and spirit of this invention.

### Examples

### Example 1

### Selection of ‘Low Human Homology’ Tags by Blast Searching

Materials and Methods:

Candidate affinity tags were screened by NCBI Blast searching against the human genome. The search was focused on sequences containing repeats of (His)4, (His)3 or (His)2 interrupted by 1 or 2 amino acids. The following criteria were used and define the term “low human homology”:
1) No human protein should have 5 or more consecutive amino acids identical to 5 or more consecutive amino acids of the tag,
AND
2) No human protein should share a window of 6 amino acids in which 5 are identical and the sixth amino acid is considered to be a conservative substitution compared to the tag.

The term “window” refers to a series of consecutive amino acids. For example, a window of 6 amino acids is a series of 6 consecutive amino acids.

The blast search was performed on the NCBI server (National Center for Biotechnology Information, NLM/NIH) with the following settings:
The algorithm blastp (protein-protein BLAST) and “search for short nearly exact matches”;
The search was done on the “nr” (non-redundant protein sequences) database;
Default options for advanced blasting were used but the organism was limited to “Homo Sapiens (taxid:9606)”;
For the identification of “conservative substitutions” the settings of Blast were used (Blastp).

Results:

Each Blast result was visually analyzed and any tag candidate with at least 5 sequential amino acids identical to a motive in a human protein was rejected. Similarly each candidate with 5 out of 6 subsequent identical amino acids and the 6^{th} amino acid being a conservative mutation (scored as a “+” in the Blast read-out) were rejected.
Example of amino acid sequence that conflicts with criterion 1):

| | |
|---|---|
| Search sequence (tag): | -HHHHH- |
| | -*****- |
| Hit sequence: | -HHHHH- |

Example of amino acid sequence that conflicts with criterium 2):

| | |
|---|---|
| Search sequence (tag): | -HHNNHH- |
| | -**+***- |
| Hit sequence: | -HHDNHH- |

The screening finally resulted in a pool of peptides fulfilling criteria 1) and 2), and are defined as ‘low human homology’ (LHH) peptides (Table 2).

TABLE 2

| sequence | SEQ ID NO |
|---|---|
| HHHWWHHH | 1 |
| HHHMWHHH | 2 |
| HHHWFHHH | 3 |
| HHWWHHWWHHWWHHWWHH | 4 |
| HHHNWHHH | 5 |
| HHWWHHWWHH | 6 |
| HHMMHHMMHH | 7 |
| HHMFHHMFHH | 8 |
| HHMWHHMWHH | 9 |
| HWHWHWHWHWH | 10 |
| HWHWHWHWHWHWH | 11 |
| HHHMFHHNWHH | 12 |
| HHMWHHHMWHHH | 13 |
| HHMFHHMFHHMFHH | 14 |
| HHMWHHHMFHHH | 15 |
| HHHMWHHHMFHHH | 16 |
| HHHWWHHHWWHHH | 17 |
| HHHWFHHHWFHHH | 18 |
| HHHMWHHHWWHHHMWHHH | 19 |
| HHHWFHHHWFHHHWFHHH | 20 |
| HHHMWHHHWWHHH | 21 |
| HHHMFHHHWWHHH | 22 |
| HHHWWHHHMWHHH | 23 |
| HHWWHHWWHHWWHH | 24 |
| HHMMHHMMHHMMHH | 25 |
| HHMWHHMWHHMWHH | 26 |
| HHHMWHHHMWHHH | 27 |
| HHMWHHHMFHHHWWHHH | 28 |
| HHHMWHHHMFHHHWWHHH | 29 |
| HHMWHHMWHHMWHHMWHH | 30 |
| HHMWHHHMWHHHMWHHH | 31 |
| HHHMFHHHWWHHHMWHHH | 32 |
| HHHWWHHHWWHHHWWHHH | 33 |
| HHHMWHHHMWHHHMWHHH | 34 |
| HHHWWHHHMWHHHWWHHH | 35 |

### Example 2

### Evaluation of LHH Tags for Binding to Ni²⁺-IMAC

Materials and Methods:

Screening by Blast searching against the human genome as mentioned in example 1 resulted in a pool of peptide sequences with low human homology (LHH).

In a next step, a random subset of candidate His-rich tag sequences, indicated with SEQ ID NO 1, SEQ ID NO 12, SEQ ID NO 13, SEQ ID N017, SEQ ID NO 22, SEQ ID NO23, was selected from this pool. Tags were produced as N-terminally biotinylated peptides (Table 3) for further evaluation in Ni-IMAC binding studies. Biotinylation of the peptides could mimic the presence of a protein on the N-terminal end of the peptides and provided—if necessary—additional tools for detection during the IMAC experiments. Between the biotin and the affinity tag sequence, a dipeptide NA linker sequence was introduced.

TABLE 3

| | Peptide | | Comprises |
|---|---|---|---|
| Peptide sequence | No | # His | SEQ ID NO |
| Bio-NA-HHHWWHHH | 3139 | 6 | 1 |
| Bio-NA-HHHWWHHHWWHHH | 3140 | 9 | 17 |
| Bio-NA-HHMWHHHMWHHH | 3142 | 8 | 13 |
| Bio-NA-HHHMFHHNWHH | 3144 | 7 | 12 |
| Bio-NA-HHHMFHHHWWHHH | 3145 | 9 | 22 |
| Bio-NA-HHHWWHHHMWHHH | 3146 | 9 | 23 |

The peptides were synthesized by solid phase synthesis and purified to >85% purity by Reverse Phase Chromatography and subsequently evaluated for binding on Ni²⁺-IMAC under denaturing conditions.

In brief, 1 mg of dry peptide powder was solubilized in 2 mL of IMAC-A buffer consisting of 50 mM phosphate, 6 M Gu.HCl, 3% n-dodecyl-N,N-dimethylglycine (also known as lauryldimethylbetaine or Empigen BB®; Albright & Wilson), pH 7.2. After solubilization, the pH of the peptide solution was verified and, if necessary, adjusted to pH 7.2 to obtain the IMAC chromatography start solution.

Further IMAC chromatography steps were executed on an Akta Purifier 10 workstation (GE healthcare Bio-Sciences). A Tricorn 5/100 column (GE healthcare Bio-Sciences) was packed with 2 mL of Ni²⁺-charged Chelating Sepharose FF resin (GE healthcare Bio-Sciences) and equilibrated with IMAC-A buffer. Next, the peptide solution was applied on the column and the column was sequentially washed with IMAC-A buffer containing 0 mM, 20 mM and 50 mM imidazole respectively till the absorbance at 280 nm reached the baseline level. Further washing and elution of the affinity tag products was performed by the sequential application of IMAC-B buffer (50 mM phosphate, 6 M Gu.HCl, pH 7.2) supplemented with 50 mM, 200 mM and 700 mM imidazole respectively till the absorbance at 280 nm reached the baseline level.

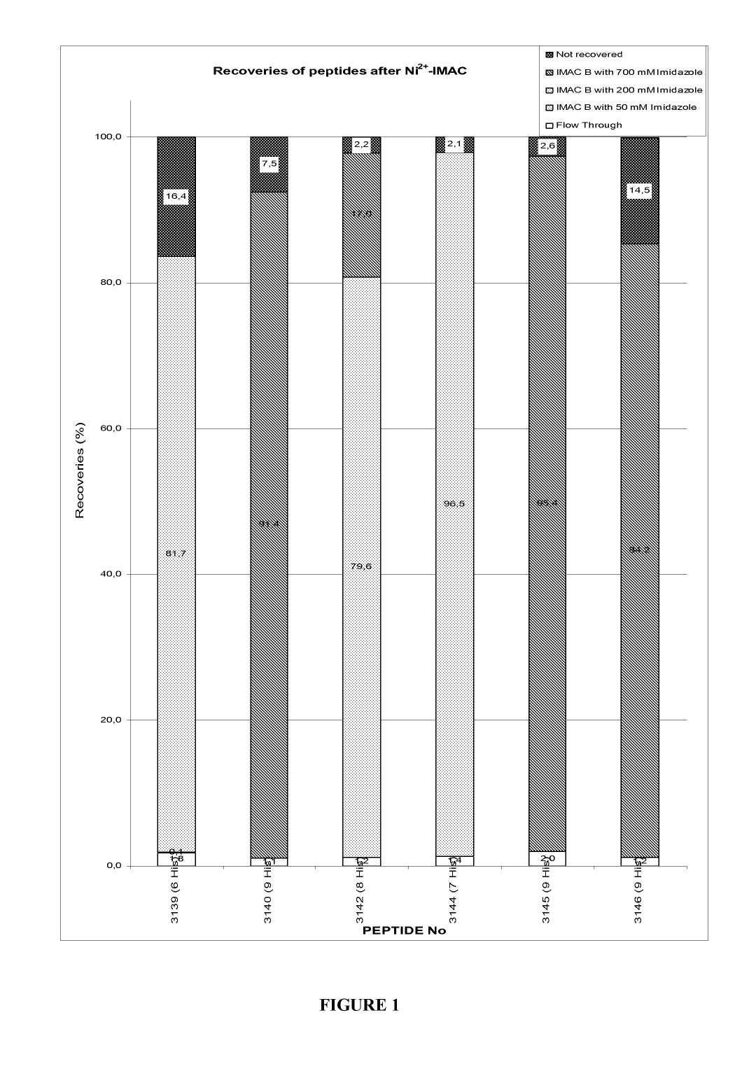

Quantification of the peptide content in the different wash and elution pools was performed by absorbance measurements at wavelengths 280 nm and 320 nm, using extinction coefficients determined from absorbance measurements of the IMAC chromatography start solutions. Alternatively, peptide concentration determination could be performed by quantification of the biotin tag, using e.g. the HABA [(2-(4′-Hydroxyazobenzene)Benzoic Acid] assay (Pierce). Peptide recoveries in the different wash and elution pools were calculated in relation to the initial peptide amount in the IMAC chromatography start solution (FIG. 1).

Results:

As shown in FIG. 1, all the tags evaluated demonstrated to be captured on the Ni²⁻-Chelating Sepharose FF resin under the chromatography conditions used. Results obtained showed also that the amount of histidine residues present in the peptide was correlated with the strength of binding.

### Example 3

### Generation of HBV and HCV Polyepitope Protein

Generation of Recombinant E. coli Strains

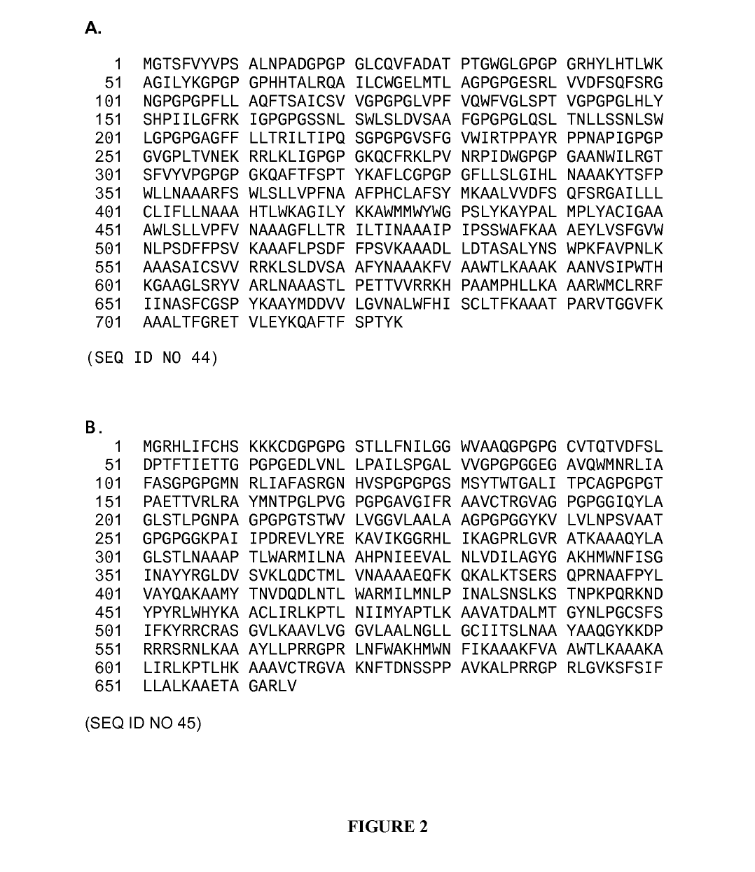

Based on the amino acid sequence of the HBV polyepitope protein (FIG. 2A—SEQ ID 44) an optimized coding sequence was designed and synthesized by GeneArt (Regensburg, Germany) using their GeneOptimizer sequence optimization software. During design, appropriate endonuclease restriction sites were introduced in the 5′ and 3′ flanking regions to simplify subcloning into the expression vectors, and an affinity tag (represented by SEQ ID NO 1, SEQ ID NO 12, SEQ ID NO 22, SEQ ID NO 31 or SEQ ID NO 32) was added preceded by a two amino acid (NA) linker sequence (Table 4). The linker sequence is selected so that the amino acid sequence obtained by said linker in combination with the neighboring protein sequence and tag sequence fulfill the low-human homology criterion as defined herein.

Based on the amino acid sequence of the HCV polyepitope protein (FIG. 2B—SEQ ID 45) an optimized coding sequence was designed and synthesized by GeneArt (Regensburg, Germany) using their GeneOptimizer sequence optimization software. During design, appropriate endonuclease restriction sites were introduced in the 5′ and 3′ flanking regions to simplify subcloning into the expression vectors, and an affinity tag (represented by SEQ ID NO 12 or SEQ ID NO 32) was added preceded by a three amino acid (NAA) linker sequence (Table 4). The linker sequence is selected so that the amino acid sequence obtained by said linker in combination with the neighboring protein sequence and tag sequence fulfill the low-human homology criterion as defined herein.

TABLE 4

| | Linker | | |
|---|---|---|---|
| | sequence | | |
| Tag | HBV | HCV | Amino acid sequence |
| LHH-03 (SEQ ID NO1) | NA | — | HHHWWHHH |
| LHH-07 (SEQ ID NO 31) | NA | — | HHMWHHHMWHHHMWHHH |
| LHH-08 (SEQ ID NO 12) | NA | NAA | HHHMFHHNWHH |
| LHH-09 (SEQ ID NO 22) | NA | — | HHHMFHHHWWHHH |
| LHH-11 (SEQ ID NO 32) | NA | NAA | HHHMFHHHWWHHHMWHHH |

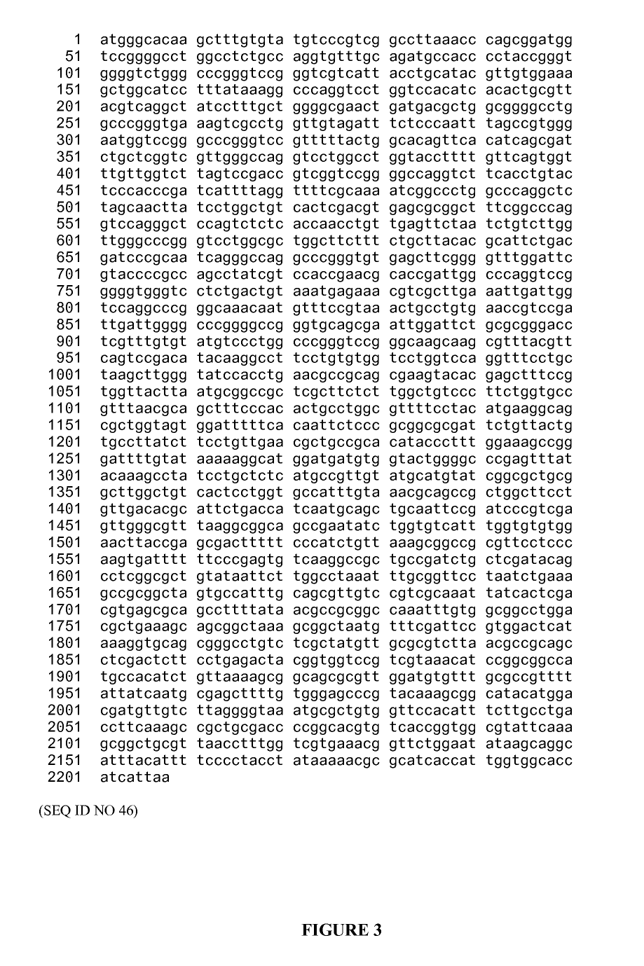

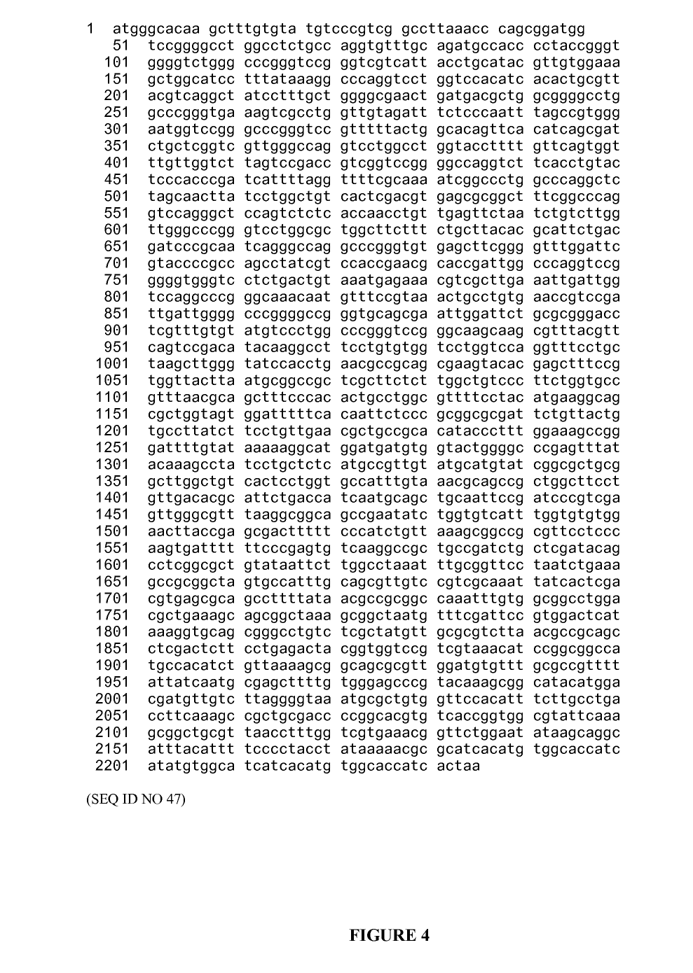

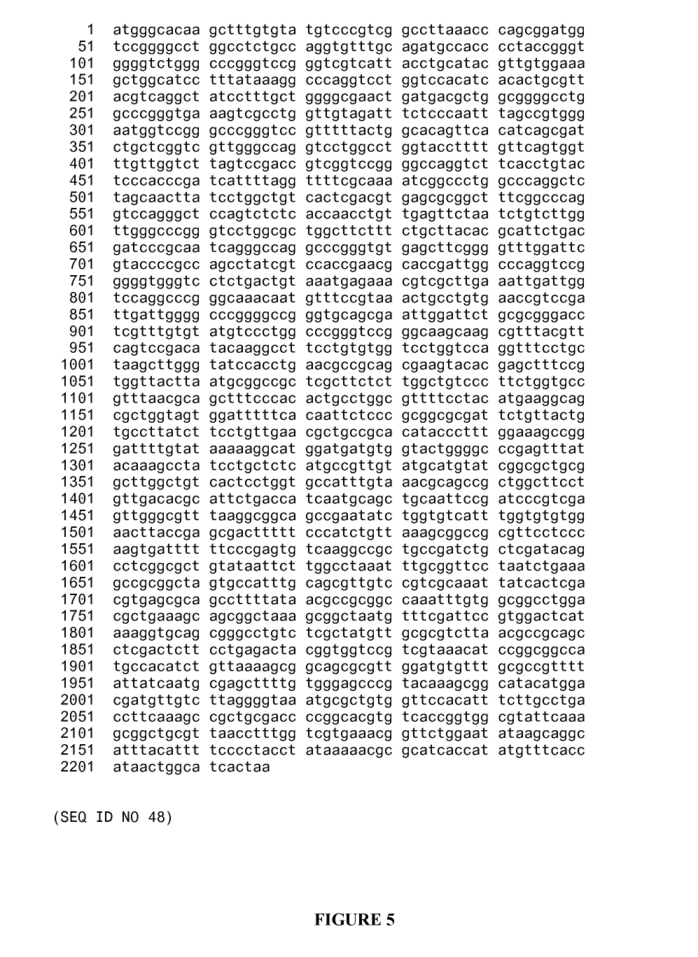

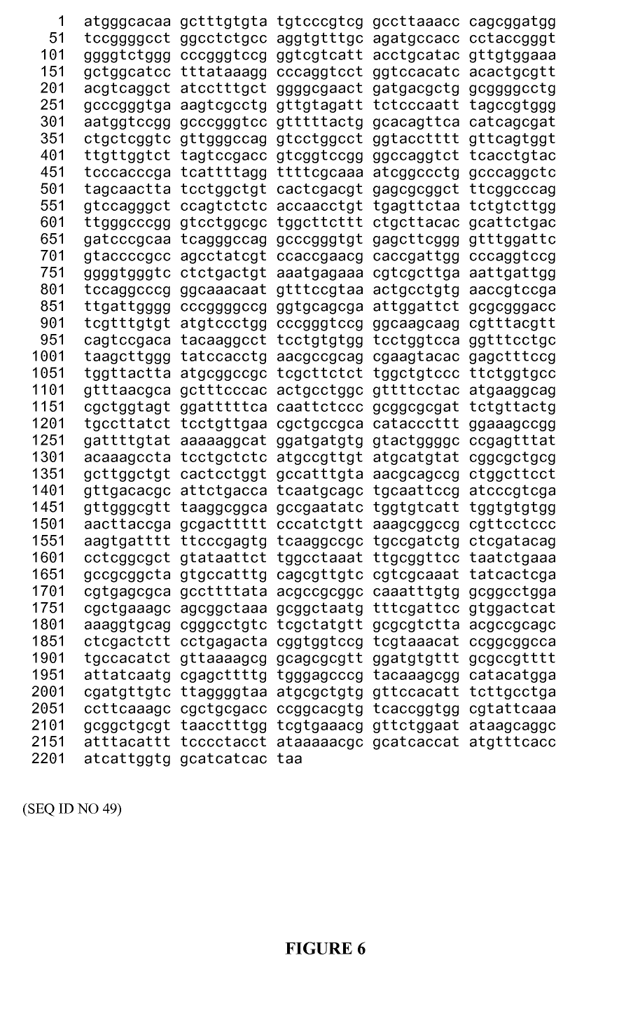

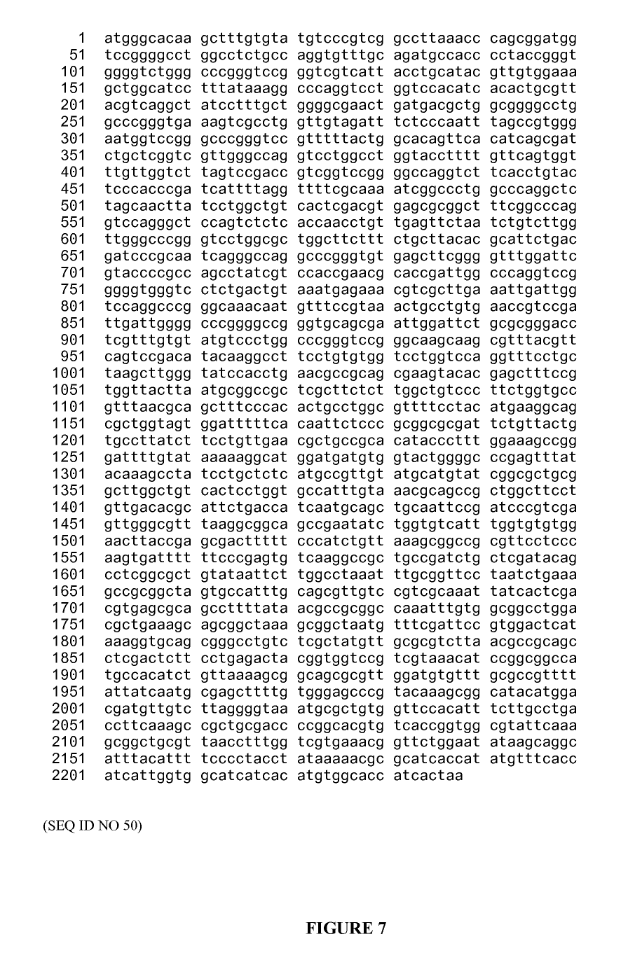

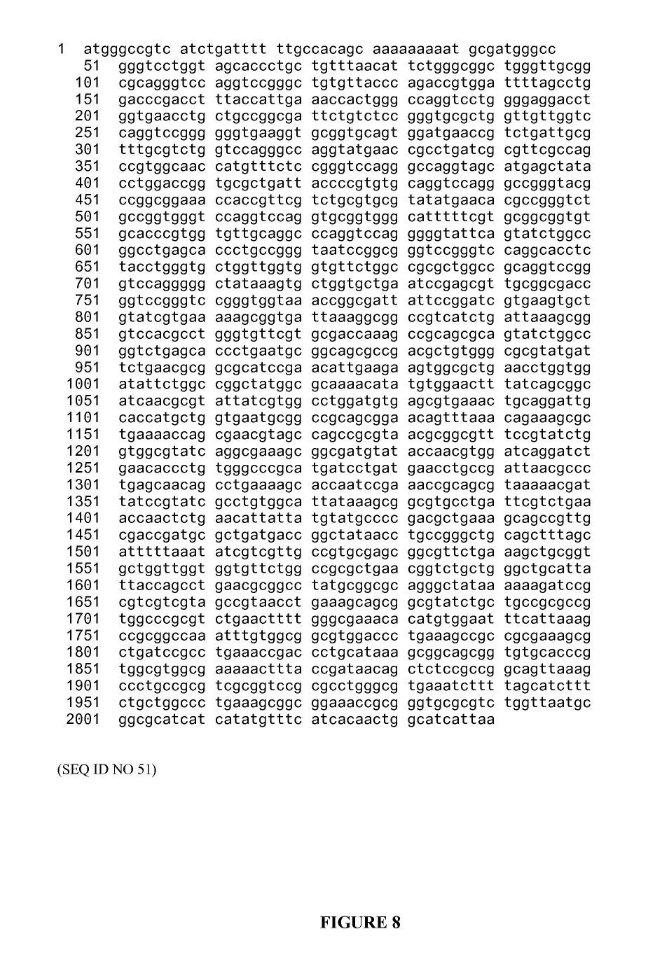

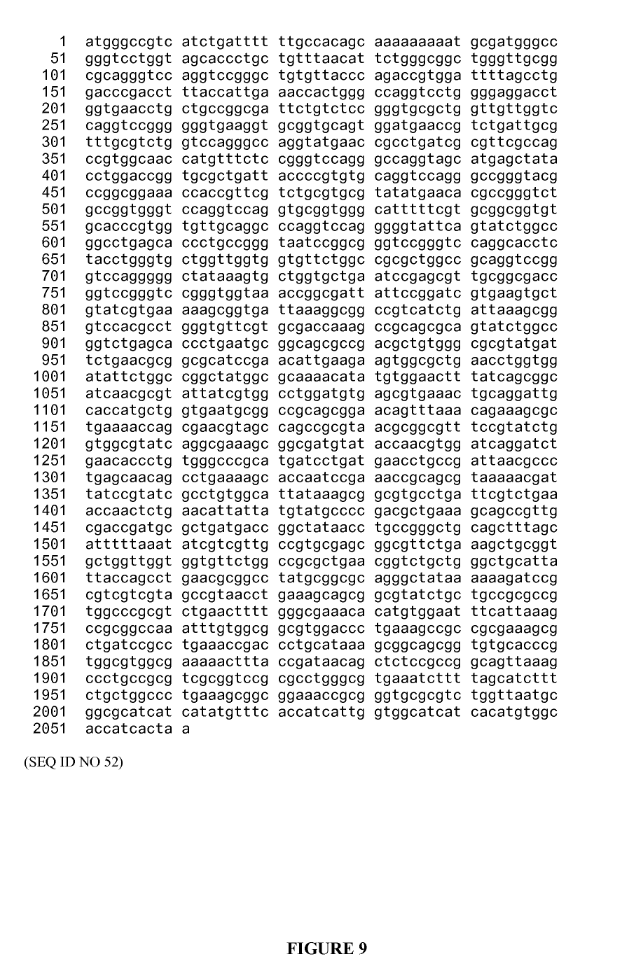

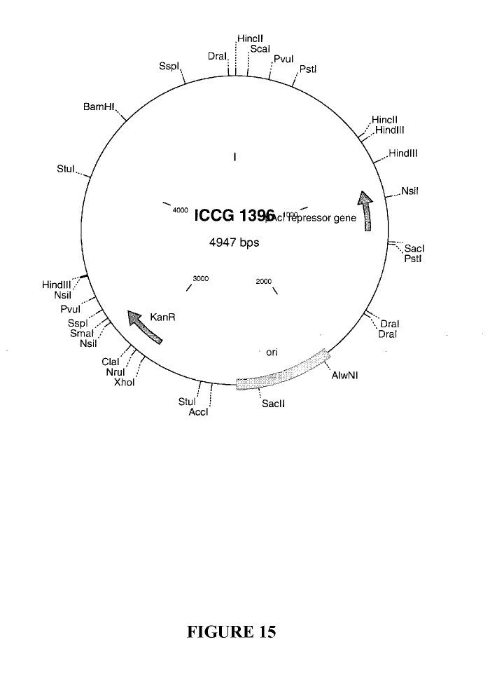

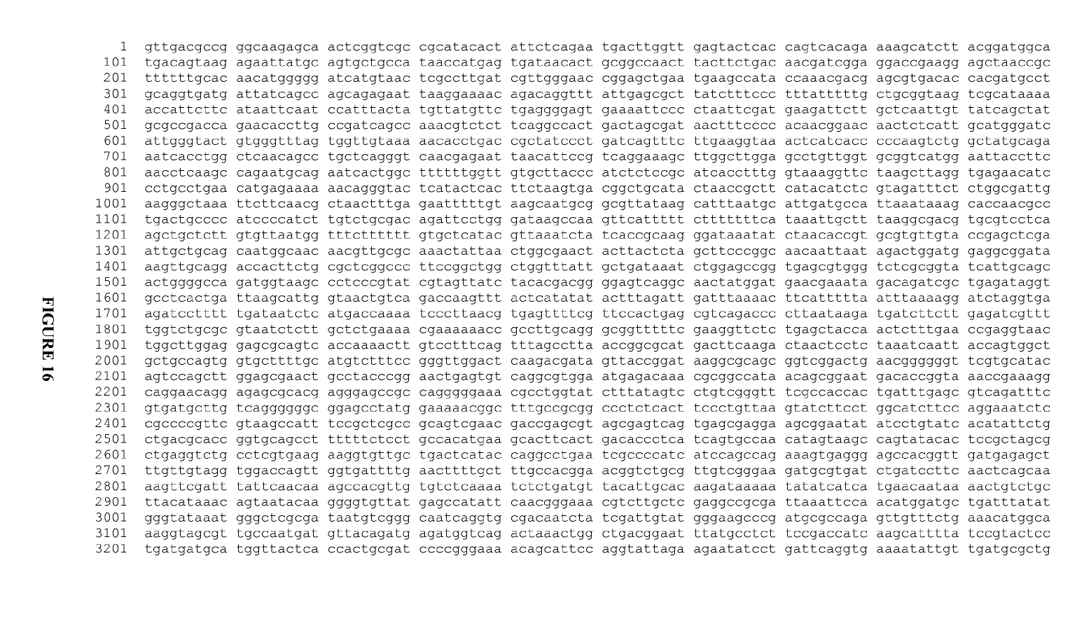

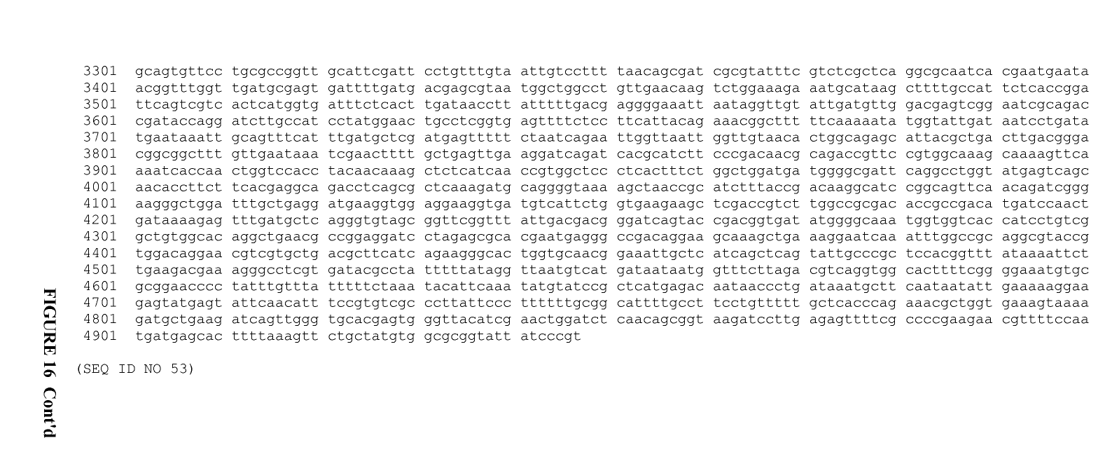

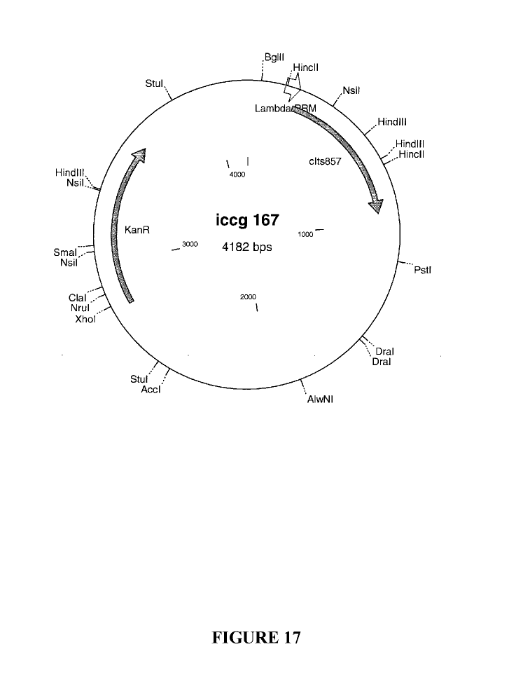

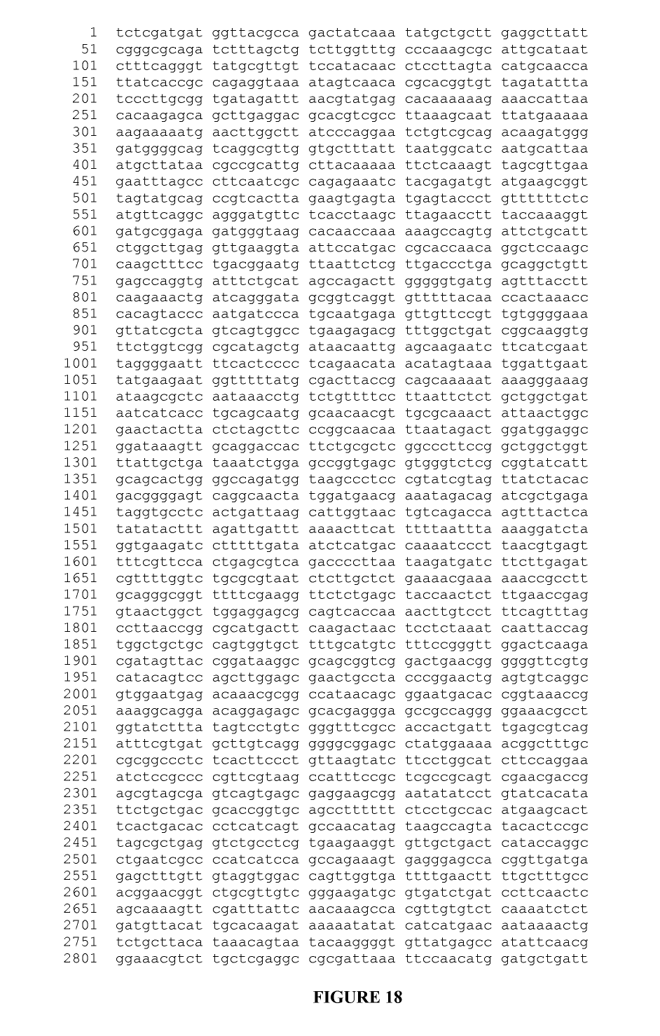

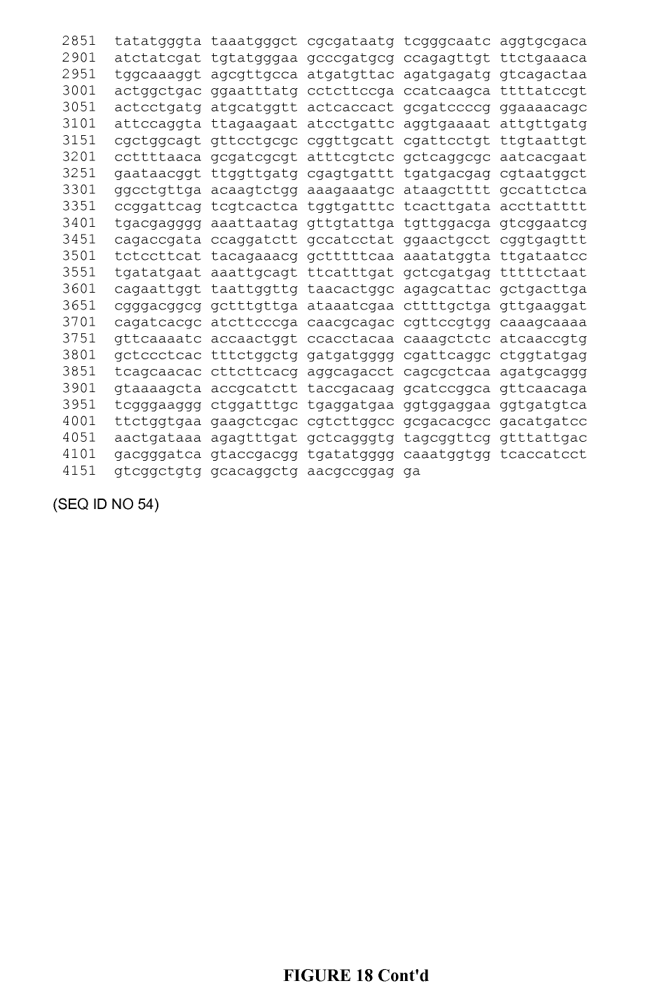

The complete HBV and HCV polyepitope coding regions (FIGS. 3 to 9—SEQ ID NO 46 to SEQ ID NO 52 respectively) were subcloned into E. coli vectors for expression using the temperature-inducible bacteriophage Lambda pR-based expression system known in the art. The final expression plasmids were transformed by a standard heat-shock method into competent E. coli host strains BL21 (Novagen, USA) and SG4044 (Gottesman et al., 1981) already transformed with resp. the plasmid pAcI (FIGS. 15-16) or plasmid pcI857 (FIGS. 17-18) ensuring the expression of the temperature-sensitive mutant of the bacteriophage Lambda cI repressor.

All subcloning was performed using standard recombinant DNA technology mainly based on the use of restriction enzymes and PCR techniques known in the art.

After transformation, individual colonies were transferred into culture medium consisting of 20 g/l of yeast extract (Becton Dickinson, ref 212750 500G), 10 g/L of tryptone (Becton Dickinson, ref. 211705 500G), 5 g/L of NaCl and 10 mg/L of tetracycline, grown at 28° C. and induced by a temperature shift to 37° C. and/or 42° C. At several time intervals up to 4 hour post induction, samples (total cell lysates) of non-induced, induced, and wild-type cells were analyzed by western blot analysis with polyclonal rabbit antisera against the HBV and HCV polyepitope protein.

Production of HBV and HCV Polyepitope Proteins in E. coli (Fermentation)

The HBV and HCV polyepitope proteins were produced from a (pre)culture in medium consisting of 20 g/l of yeast extract (Becton Dickinson, ref 212750 500G), 10 g/L of tryptone (Becton Dickinson, ref. 211705 500G), 5 g/L of NaCl and 10 mg/L of tetracycline.

Preculture medium (500 mL in 2 L baffled shake flasks) was inoculated with 500 μL from a cell bank glycerol slant. Precultures were incubated at 28° C. and 200-250 rpm for 22 to 24 h. Baffled shake flasks (2 L) were filled with 500 mL of culture medium and inoculated 1/20 (v/v) with preculture broth. The culture was allowed to grow for 4 h at 28° C. and was induced for 3 h at 37° C. Cells were recovered from the culture broth by centrifugation in a Beckman JLA10.500 rotor at 9000 rpm at 4° C. for 25 min. Cell pellets were stored at −70° C.

### Example 4

### Evaluation of Tags for Use in IMAC-Purification of Fusion Constructs

A. HBV Polyepitope Fusion Constructs

Materials and Methods:

Ni²⁺-IMAC capture and intermediate purification performance was evaluated for the different

HBV fusion proteins (i.e. proteins encoded by the nucleic acid sequences represented by SEQ ID NO 46, SEQ ID NO 47, SEQ ID NO 48, SEQ ID NO 49, and SEQ ID NO 50) under denaturing conditions, after cell disruption, inclusion body harvest/extraction, Gu.HCl-solubilization, disulphide bridge disruption, reversible cystein blocking and clarification.

In brief, cell pellets obtained from 400 mL cultures were resuspended in 5 volumes (5 mL buffer/gram wet weight cell pellet) of lysis buffer (50 mM Tris/HCl, pH 8.0) to which 2 mM MgCl₂, 1/25 Complete from 25× stock solution and 10 U/mL benzonase purity grade II was added. After homogenization using a Polytron PT1200 (Kinematica AG), cell disruption was performed by sonication using a Soniprep 150 device (Serlabo) (9 cycli: 20 seconds ON, 40 seconds OFF). Samples were incubated on ice during sonication.

Cell lysates obtained were subjected to centrifugation (18.500 g for 1 hour at 4° C.) and pellet fraction was recovered. The pellet was resuspended in 3 volumes (3 mL buffer/gram wet weight original cell pellet) of inclusion body wash buffer I (0.25 M Gu.HCl, 10 mM EDTA, 10 mM DTT, 1% sodium N-lauroylsarcosinate, 50 mM Tris/HCl pH 8.0) and stirred for 45 minutes at 20° C., followed by centrifugation (18.500 g for 30 minutes at 4° C.). After discarding the supernatans, the pellet was collected and resuspended in 1.5 volumes (1.5mL buffer/gram wet weight original cell pellet) of inclusion body wash buffer II (10 mM EDTA, 10 mM DTT, 1% Triton X-100, 50 mM Tris/HCl pH 8.0). After stirring for 30 minutes at 20° C., suspension was centrifuged (18.500 g for 30 minutes at 4° C.). Next, pellet fraction obtained was subjected to a third inclusion body wash step by resuspension in 1.5 volumes (1.5 mL buffer/gram wet weight original cell pellet) of inclusion body wash buffer III (50 mM Tris/HCl buffer, pH 8.0, to which 5 mM MgCl₂, 1% Triton X-100 and 10 U/mL benzonase purity grade II was added) followed by stirring for 30 minutes at 20° C. and subsequent centrifugation at 18.500 g for 30 minutes (4° C.). After recovery of the inclusion body (IB) pellet, the fusion protein was extracted from the IB-pellet by resuspending in 9 mL extraction buffer (6.7M Gu.HCl, 56 mM Na₂HP0₄.2H₂0, pH 7.2) per gram inclusion body pellet (wet weight) and subsequent stirring for 60 minutes at 20° C. Then, the protein solution was clarified by centrifugation (18.500 g for 30 minutes at 4° C.).

Soluble protein in the supernatant was sulfonated by addition of sodium sulfite, sodium tetrathionate and L-cystein to final concentrations of respectively 320 mM, 65 mM and 0.2 mM. After subsequent pH adjustment to pH 7.2, protein solution was stirred overnight at room temperature in contact with air and shielded from the light. Then, n-dodecyl-N,N-dimethylglycine (also known as lauryldimethylbetaine or Empigen BB®, Albright & Wilson) and imidazole were added to the protein solution to a final concentration of 3% (w/v) and 20 mM respectively and the pH was adjusted to pH 7.2. The sample was filtrated through a 0.22 μm pore size bottle top filter with prefilter (Millipore).

All further chromatographic steps were executed on an Äkta Purifier 10 workstation (GE healthcare Bio-Sciences). A Tricorn 10/100 column (GE healthcare Bio-Sciences) was packed with 7.8 mL of Ni²⁺-charged Chelating Sepharose FF resin (GE healthcare Bio-Sciences) and equilibrated with 50 mM phosphate, 6 M Gu.HCl, 3% Empigen BB®, pH 7.2 (IMAC-A buffer) supplemented with 20 mM imidazole.

Next, the protein sample was loaded on the column. The column was washed sequentially with IMAC-A buffer containing 20 mM and 50 mM imidazole respectively till the absorbance at 280 nm reached the baseline level. Further washing and elution of the fusion proteins was performed by the sequential application of IMAC-B buffer (50 mM phosphate, 6 M Gu.HCl, pH 7.2) supplemented with 50 mM imidazole, 200 mM imidazole and 700 mM imidazole respectively till the absorbance at 280 nm reached the baseline level.

All protein fractions obtained were analyzed by SDS-PAGE analysis under non-reducing conditions (+subsequent silver staining) and western-blotting using polyclonal rabbit antisera directed against the HBV fusion protein that were pre-incubated with E. coli lysate (MC 1061(pAcI)+BL21 (pAcI)).

Protein concentration in the 200 mM and 700 mM imidazole IMAC elution pools was determined by measuring absorbance at 280 nm and subtraction of the absorbance at 320 nm, assuming that a protein solution of 1 mg/mL in a cuvette with 1 cm optical pathlength yields an absorbance at 280 nm of 2.2.

Results:

Western blot analysis confirmed an efficient capture efficiency (>80%) under the chromatography conditions used for all fusion constructs studied.

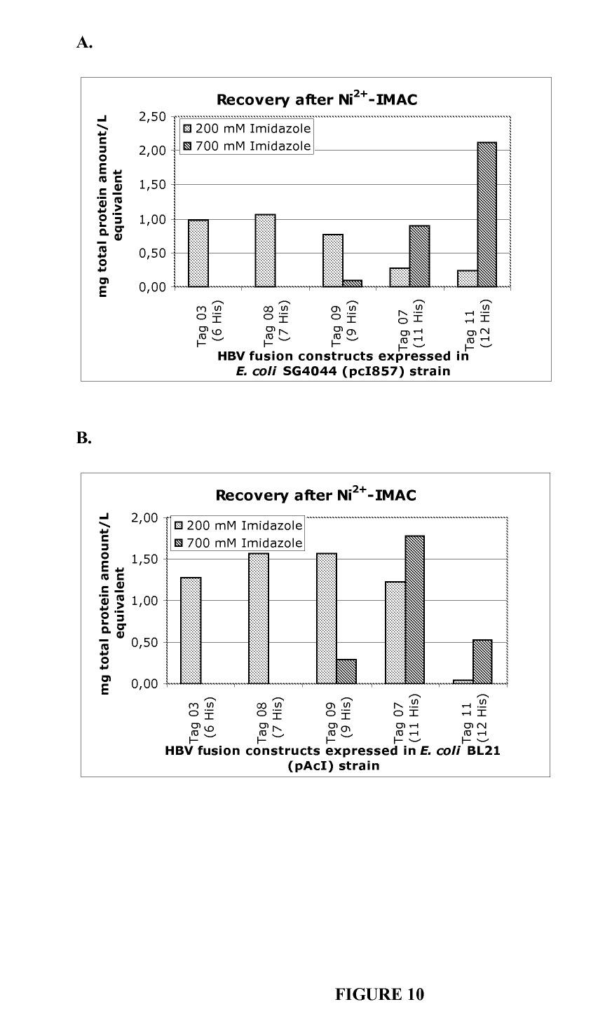

Protein quantification in the elution pools by absorbance measurements (FIGS. 10A, 10B) showed that the amount of histidine residues present in the tag was correlated with the strength of binding on the IMAC resin. The fusion constructs with tags SEQ ID NO 1 and SEQ ID NO 12 containing respectively 6 and 7 histidines in the tag, eluted at a 200 mM imidazole concentration, whereas the fusion constructs with tags SEQ ID NO 22, SEQ ID NO 31 and SEQ ID NO 32 containing respectively 9, 11 and 12 histidines in the tag eluted at 200 mM and 700 mM imidazole. Only a minority of the fusion constructs with tag SEQ ID NO 32 was eluted at 200 mM imidazole.

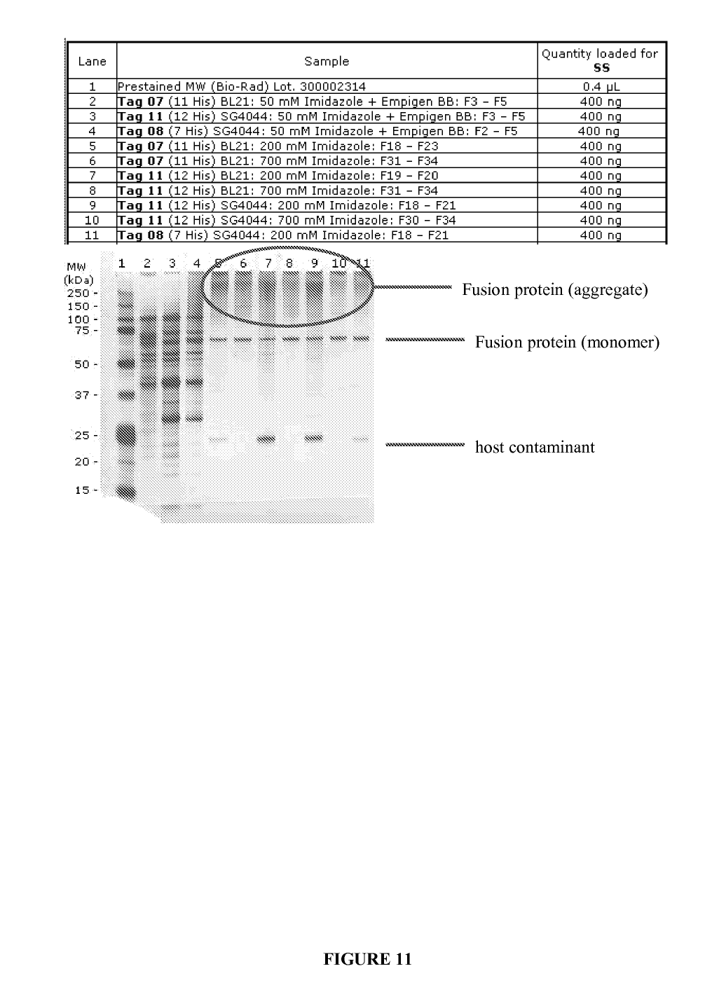

SDS-PAGE and subsequent silver staining of the IMAC-protein fractions showed that, under the purification conditions used, protein purities of >80% could be obtained for all different fusion constructs in respectively the 200 mM or 700 mM imidazole elution pools (FIG. 11). A host cell protein band of ˜25 kDa was still observed on SDS-PAGE gel in the 200 mM imidazole elution pools (FIG. 11). No residual contamination was observed in the 700 mM imidazole elution pools (FIG. 11). Therefore, use of tags resulting in increased affinity on IMAC (i.e. resulting in negligible elution at 200 mM imidazole conditions) provide a superior purification tool compared to the traditional hexahistidine metal affinity tag, enabling removal of histidine-rich host contaminants (e.g. SlyD) in a stringent washing step, prior to efficient recovery of the fusion protein at higher imidazole concentrations.

B. HCV Polyepitope Fusion Constructs

Materials and Methods:

Ni²⁺-IMAC capture and intermediate purification performance was evaluated for the HCV fusion constructs (i.e. proteins encoded by the nucleic acid sequence represented by SEQ ID NO 51 and SEQ ID NO 52) under denaturing conditions, after cell disruption, inclusion body harvest/extraction, Gu.HCl-solubilization, disulphide bridge disruption, reversible cystein blocking and clarification.

In brief, cell pellets obtained from 800 mL cultures were resuspended in 5 volumes (5 mL buffer/gram wet weight cell pellet) of lysis buffer (50 mM Tris/HCl, pH 8.0) to which 2 mM MgCl₂, 1/25 Complete from 25× stock solution and 10 U/mL benzonase purity grade II was added. After homogenization using a Polytron PT1200 (Kinematica AG), cell disruption was performed by sonication using a Soniprep 150 device (Serlabo) (9 cycli: 20 seconds ON, 40 seconds OFF). Samples were incubated on ice during sonication.

Cell lysates obtained were subjected to centrifugation (18.500 g for 1 hour at 4° C.) and pellet fraction was recovered. The pellet was resuspended in 3 volumes (3 mL buffer/gram wet weight original cell pellet) of inclusion body wash buffer I (0.25 M Gu.HCl, 10 mM EDTA, 10 mM DTT, 1% sodium N-lauroylsarcosinate, 50 mM Tris/HCl pH 8.0) and stirred for 45 minutes at 20° C., followed by centrifugation (18.500 g for 30 minutes at 4° C.). After discarding the supernatans, the pellet was collected and resuspended in 1.5 volumes (1.5 mL buffer/gram wet weight original cell pellet) of inclusion body wash buffer II (10 mM EDTA, 10 mM DTT, 1% Triton X-100, 50 mM Tris/HCl pH 8.0). After stirring for 30 minutes at 20° C., suspension was centrifuged (18.500 g for 30 minutes at 4° C.). Next, pellet fraction obtained was subjected to a third inclusion body wash step by resuspension in 1.5 volumes (1.5 mL buffer/gram wet weight original cell pellet) of inclusion body wash buffer III (50 mM Tris/HCl buffer, pH 8.0, to which 5 mM MgCl₂, 1% Triton X-100 and 10 U/mL benzonase purity grade II was added) followed by stirring for 30 minutes at 20° C. and subsequent centrifugation at 18.500 g for 30 minutes (4° C.). After recovery of the inclusion body (IB) pellet, HCV fusion protein was extracted from the IB-pellet by resuspending in 9 mL extraction buffer (6.7M Gu.HCl, 56 mM Na₂HP0₄.2H₂0, pH 7.2) per gram inclusion body pellet (wet weight) and subsequent stirring for 60 minutes at 20° C.

Then, the protein solution was clarified by centrifugation (18.500 g for 30 minutes at 4° C.). Soluble protein in the supernatant was sulfonated by addition of sodium sulfite, sodium tetrathionate and L-cystein to final concentrations of respectively 320 mM, 65 mM and 0.2 mM. After subsequent pH adjustment to pH 7.2, protein solution was stirred overnight at room temperature in contact with air and shielded from the light. Then, n-dodecyl-N,N-dimethylglycine (also known as lauryldimethylbetaine or Empigen BB®, Albright & Wilson) and imidazole were added to the protein solution to a final concentration of 3% (w/v) and 20 mM respectively and the pH was adjusted to pH 7.2. The sample was filtrated through a 0.22 μm pore size bottle top filter with prefilter (Millipore).

All further chromatographic steps were executed on an Äkta Purifier 10 workstation (GE healthcare Bio-Sciences). A Tricorn 10/100 column (GE healthcare Bio-Sciences) was packed with 7.8 mL of Ni²⁺-charged Chelating Sepharose FF resin (GE healthcare Bio-Sciences) and equilibrated with 50 mM phosphate, 6 M Gu.HCl, 20 mM imidazole, pH 7.2 (IMAC-C buffer) supplemented with 3% Empigen BB®.

Next, the protein sample was loaded on the column. The column was washed sequentially with IMAC-C buffer containing 3% Empigen BB® and IMAC-C buffer without 3% Empigen BB® till the absorbance at 280 nm reached the baseline level. Further washing and elution of fusion products was performed by the sequential application of IMAC-D buffer (20 mM Tris, 8 M urea, pH 7.2) supplemented with 20 mM imidazole, 50 mM imidazole, 200 mM imidazole and 700 mM imidazole respectively till the absorbance at 280 nm reached the baseline level.

All protein fractions obtained were analyzed by SDS-PAGE analysis under non-reducing conditions (+subsequent Coomassie staining) and western-blotting using a mouse monoclonal antibody directed against the tags, that was pre-incubated with E. coli lysate (MC 1061(pAcI)+BL21 (pAcI)).

Protein concentration in the 200 mM and 700 mM imidazole IMAC elution pools was determined by measuring absorbance at 280 nm and subtraction of the absorbance at 320 nm, assuming that a protein solution of 1 mg/mL in a cuvette with 1 cm optical pathlength yields an absorbance at 280 nm of 1.4.

Results:

Western blot analysis on equivalent amounts of IMAC start and IMAC flow-through pools confirmed an efficient capture efficiency (>80%) under the chromatography conditions used for the fusion constructs.

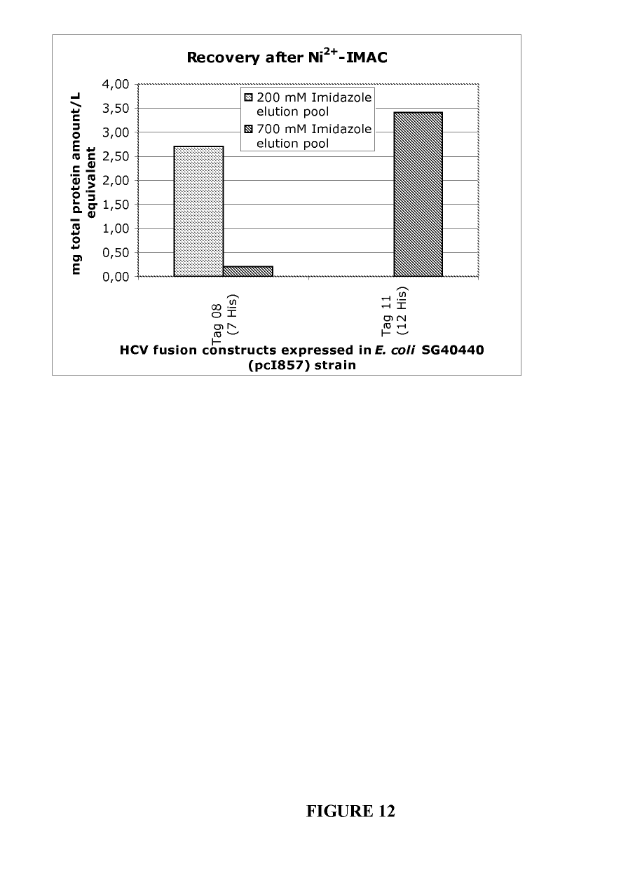

Protein quantification in the elution pools by absorbance measurements (FIG. 12) also confirmed that the amount of histidine residues present in the tag was correlated with the strength of binding on the IMAC resin. The fusion construct with the tag (SEQ ID NO 12) containing 7 histidines in the tag, eluted at a 200 mM imidazole concentration, whereas the fusion construct with the tag (SEQ ID NO 32) containing 12 histidines in the tag eluted at 700 mM imidazole.

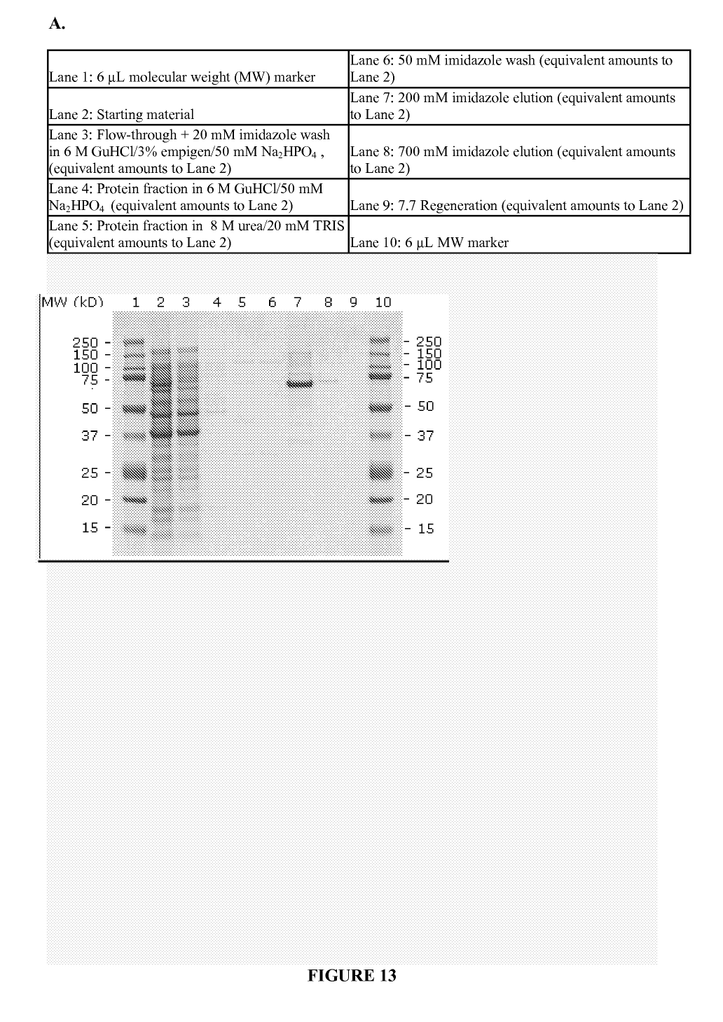

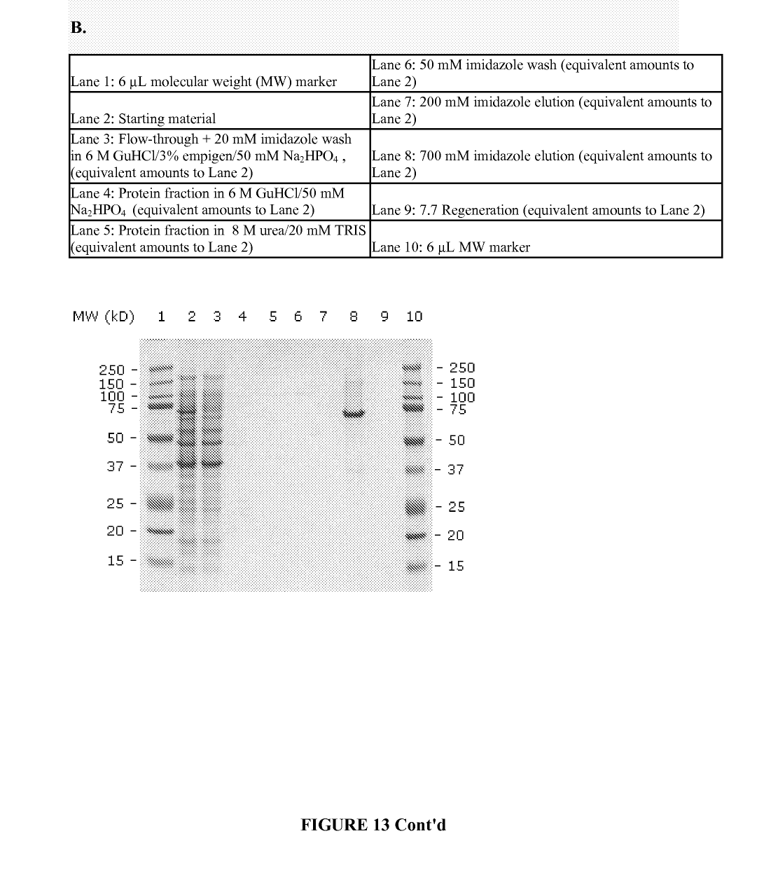

SDS-PAGE and subsequent Coomassie staining of the IMAC-protein fractions showed that, under the purification conditions used, protein purities of >85% could be obtained for both tag fusion constructs in respectively the 200 mM or 700 mM imidazole elution pools (FIGS. 13A, 13B).

### Example 5

### Evaluation of Tags for Use in IMAC-Purification of Fusion Constructs Without Preceding Enrichment by Inclusion Body Washing Steps

Materials and Methods:

Ni²⁺-IMAC capture and intermediate purification performance was evaluated for the fusion construct encoded by SEQ ID NO 52 under denaturing conditions, after cell disruption by Gu.HCl-solubilization and disulphide bridge disruption, reversible cystein blocking and clarification.

In brief, cell pellet obtained from 2.7 L culture was resuspended in 10 volumes (10 mL buffer/gram wet weight cell pellet) of lysis buffer (6M Gu.HCl, 50 mM Na₂HP0₄.2H₂0, pH 7.2) and sodium sulfite, sodium tetrathionate and L-cystein were added to final concentrations of respectively 320 mM, 65 mM and 0.2 mM. After subsequent pH adjustment to pH 7.2, solution was stirred overnight at room temperature in contact with air and shielded from the light. The cell lysate obtained was clarified by centrifugation (18.500 g for 60 minutes at 4° C.). Pellet was discarded and the supernatant, containing the soluble fusion protein fraction, was recovered. Then, n-dodecyl-N,N-dimethylglycine (also known as lauryldimethylbetaine or Empigen BB®, Albright & Wilson) and imidazole were added to the protein solution to a final concentration of 3% (w/v) and 20 mM respectively and the pH was adjusted to pH 7.2. The sample was filtrated through a 0.22 μm pore size bottle top filter with prefilter (Millipore).

All further chromatographic steps were executed on an Akta Explorer 100 workstation (GE healthcare Bio-Sciences). A XK 16/20 column (GE healthcare Bio-Sciences) was packed with 20 mL of Ni²⁺-charged Chelating Sepharose FF resin (GE healthcare Bio-Sciences) and equilibrated with 50 mM phosphate, 6 M Gu.HCl, 20 mM imidazole, pH 7.2 (IMAC-E buffer) supplemented with 3% Empigen BB®.

Next, the protein sample was loaded on the column. The column was washed sequentially with IMAC-E buffer containing 3% Empigen BB® and IMAC-E buffer without 3% Empigen BB® till the absorbance at 280 nm reached the baseline level. Further washing and elution of the fusion product was performed by the sequential application of IMAC-F buffer (20 mM Tris, 8 M urea, pH 7.2) supplemented with 20 mM imidazole, 50 mM imidazole, 200 mM imidazole and 700 mM imidazole respectively till the absorbance at 280 nm reached the baseline level.

All protein fractions obtained were analyzed by SDS-PAGE analysis under non-reducing conditions (+subsequent silver staining) and western-blotting using for specific detection, polyclonal rabbit antisera directed against the HCV fusion protein that were pre-incubated with E. coli lysate (MC 1061 (pAcI)+BL21 (pAcI)).

Protein concentration in the 200 mM and 700 mM imidazole IMAC elution pools was determined by measuring absorbance at 280 nm and subtraction of the absorbance at 320 nm, assuming that a protein solution of 1 mg/mL in a cuvette with 1 cm optical pathlength yields an absorbance at 280 nm of 1.5.

Results:

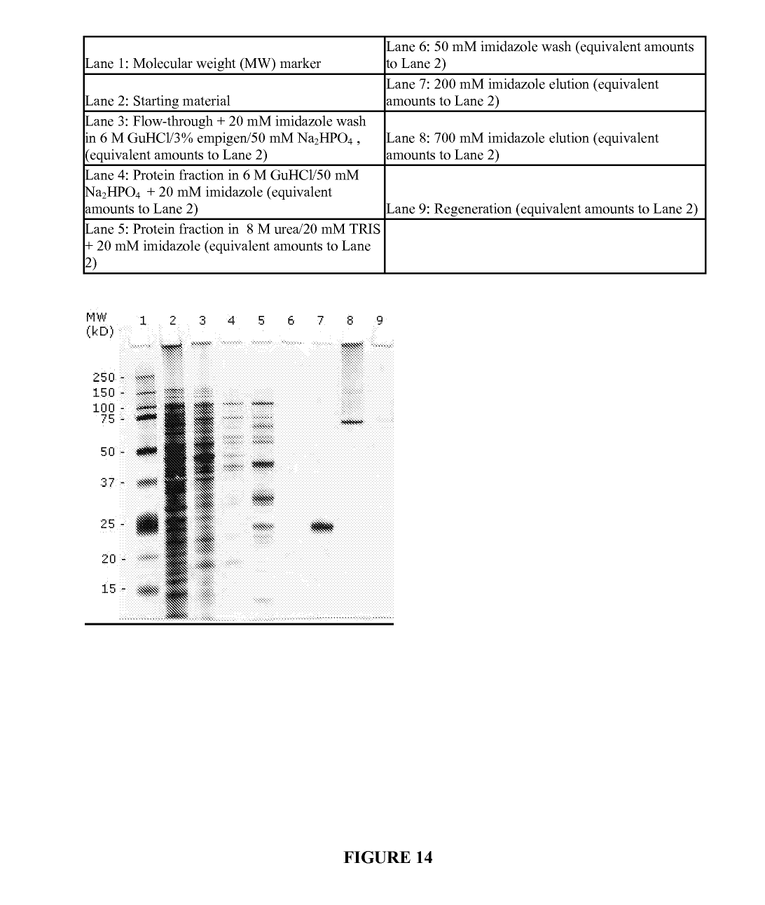

Despite the use of a more stringent disruption/solubilization procedure and abolishment of inclusion body isolation and inclusion body washing steps—earlier used for efficient removal of large amounts of host contaminants—the fusion protein was mainly recovered in the 700 mM imidazole fraction with >90% purity. No host cell protein bands (also not around ˜25 kDa) were observed on SDS-PAGE gel in the 700 mM imidazole elution pool (FIG. 14). Removal of histidine-rich host contaminants (e.g. SlyD) was accomplished in the 200 mM imidazole washing (FIG. 14). This confirmed that use of LHH-tags resulting in increased affinity on

IMAC (i.e. resulting in negligible elution at 200 mM imidazole conditions) provide a superior purification tool compared to the traditional hexahistidine metal affinity tag, enabling removal of histidine-rich host contaminants (e.g. SlyD) in a stringent washing step, prior to efficient recovery of the fusion protein at higher imidazole concentrations.

### Example 6

### LHH-Tagged Proteins Do Not Induce High Titer Responses to the LHH-Tag

To evaluate the immunogenicity of LHH-tagged (HTL-CTL)_HBV or (HTL-CTL)_HCV proteins, heterologous prime-boost immunizations will be administrated in mice. Mouse serum was collected and the humoral anti LHH responses were evaluated using the LHH-11 peptide (IGP3147) and/or the LHH-8 peptide (IGP3144) in a peptide coating ELISA (Table 5). For comparison, the antibody reactivity against the HCV or HBV poly-epitope protein was measured in a protein coating ELISA.

TABLE 5

| | | Pep- | |
|---|---|---|---|
| | Peptide | tide | Comprises |
| Peptide sequence | No | Ref. | SEQ ID NO |
| Bio-NA-HHHMFHHNWHH | 3144 | LHH-8 | 12 |
| Bio-NA-HHHMFHHHWWHHHMWHHH | 3147 | LHH-11 | 32 |

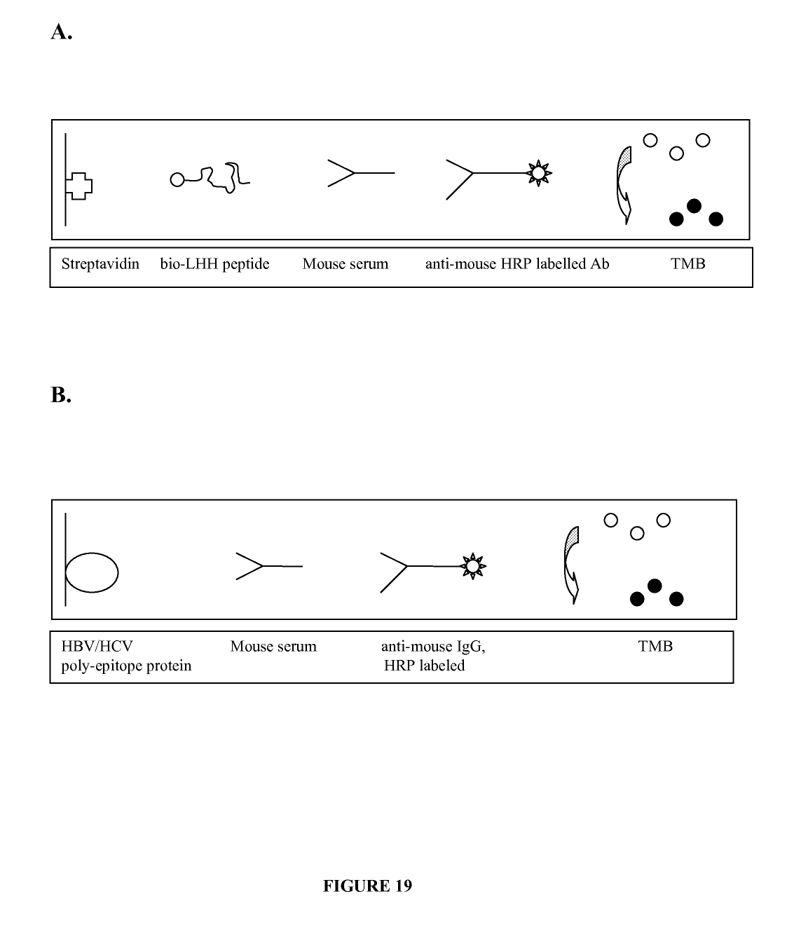

LHH Peptide Coating ELISA (FIG. 19A)

All incubation steps were performed with a volume of 0.1 ml per well. After each incubation step the microtitre plates were emptied and washed three to five times with PBS-Tween.

The biotinylated LHH-peptides, LHH-8 (IGP3144) and/or LHH-11 (IGP3147) were incubated on a streptavidin pre-coated plate at a concentration of 1 μg/ml for 2 h at room temperature. The mouse sera were added in a ⅓ serial dilution in assay diluent starting at a 1/100 dilution and incubated for 1 h at 37° C. As blank value 100 μl assay diluent was used. The bound anti LHH antibodies were detected with a HRP-labelled anti mouse antibody ( 1/20000 in assay diluent).

The plate was incubated for 1 h at 37° C.

The addition of TMB ( 1/100 in substrate buffer) for 30 minutes at room temperature resulted in color development proportional with the concentration of bound antibodies. The color reaction was stopped with 50 μl/well 2N H₂SO₄. The plate was read at 450-595 or 450 nm.

HBV or HCV Protein Coating ELISA (FIG. 19B)

All incubation steps were performed with a volume of 0.1 ml per well, expect for the blocking solution (0.2 ml). After each incubation step the microtitre plates were emptied and washed one to five times with PBS-Tween.

The (HTL-CTL)_HBV or (HTL-CTL)_HCV proteins were coated overnight at 4° C. in 10.10 buffer. For the LHH tagged (HTL-CTL)_HBV proteins the coating concentration was 10 μg/ml. The (his)6 tagged (HTL-CTL)_HBV protein and the LHH-11 tagged (HTL-CTL)_HCV protein were coated at 5 μg/ml. After blocking the plates with assay dilutent the mouse sera were incubated for 1 h at 37° C. on the coated proteins in a ⅓ serial dilution, starting at a 1/100 dilution. As blank value 100 μl assay diluent was used. The bound anti HCV or HBV poly-epitope antibodies were detected with a HRP-labeled anti-mouse antibody ( 1/20000 in assay diluent) and incubated for 1 h at 37° C. The addition of TMB ( 1/100) resulted in color development proportional with concentration of bound antibodies. After 30 minutes of TMB incubation the reaction was stopped with 2N H₂SO₄. The plate was read at 450-595 or 450 nm.

Data Analysis

‘Titer’ was determined as the serum dilution factor with an OD value higher than 2× (average OD values of the control samples), also defined as the cut-off value. For sera with an OD value lower than the cut-off value at the 1/100 dilution, titers will be marked <100.

Result

LHH Tagged (HTL-CTL) HBV Protein (TR RDTX AD 21):

Mouse sera were serially diluted on IGP 3144 (LHH-8) and IGP 3147 (LHH-11) peptides. This sera were also tested on three (HTL-CTL)_HBV poly-epitope proteins, only differing in tag (LHH-11, LHH-8 or (his)6 tag).

Titers were calculated for each serial dilution as summarized in Table 6. Sera marked as ‘>72900’ were not completely diluted at a 1/72900 dilution and would probably dilute another 1 or 2 dilutions before reaching OD values below the cut-off.

For only one mouse (8923), weak reactivity was seen against the LHH-tag, indicating that the LHH-11/LHH-8 tagged proteins hardly induce LHH-specific antibodies.

Serum from mouse 8923 showed a different reactivity profile than all the other mice. Reactivity against the LHH-8 tagged (HTL-CTL)_HBV poly-epitope protein was stronger compared with poly-epitope proteins with an LHH11 or (his)6 tag. This mouse also recognized the LHH-8 peptide in contrast with the other mice, explaining the stronger reactivity against the LHH-8 tagged protein (anti-LHH8+anti-poly-epitope protein reactivity).

TABLE 6
The antibody titers of the mouse sera tested in the peptide and protein coating ELISA

| | Mouse | HBV-HTL_CTL | | | IGP 3144 | IGP 3147 |
|---|---|---|---|---|---|---|
| Antigen | ID | _(His)6 | _LHH-8 | _LHH-11 | LHH-8 | LHH-11 |
| LHH-11 tagged (HTL-CTL)_HBV | 9391 | >72900 | >72900 | >72900 | <100 | <100 |
| | 9392 | 72900 | 72900 | 72900 | <100 | <100 |
| | 9393 | 72900 | 72900 | 72900 | <100 | <100 |
| | 9394 | 24300 | 24300 | 24300 | <100 | <100 |
| | 9396 | 8100 | 8100 | 8100 | <100 | <100 |
| | 9397 | 72900 | 72900 | 72900 | <100 | <100 |
| | 9398 | 24300 | 24300 | 24300 | <100 | <100 |
| | 9399 | 72900 | 72900 | 72900 | <100 | <100 |
| | 9400 | >72900 | >72900 | >72900 | <100 | <100 |
| | 8922 | 8100 | 24300 | 8100 | <100 | <100 |
| | 9401 | 72900 | 72900 | 24300 | <100 | <100 |
| | 9402 | 24300 | 24300 | 24300 | <100 | <100 |
| | 9403 | >72900 | >72900 | >72900 | <100 | <100 |
| | 9404 | 24300 | 24300 | 72900 | <100 | <100 |
| | 9405 | 72900 | 72900 | 72900 | <100 | <100 |
| | 9406 | 24300 | 24300 | 24300 | <100 | <100 |
| LHH-8 tagged (HTL-CTL)_HBV | 9407 | 8100 | 2700 | 8100 | <100 | <100 |
| | 9408 | 72900 | 24300 | 72900 | <100 | <100 |
| | 9409 | 24300 | 2700 | 8100 | <100 | <100 |
| | 9410 | 24300 | 24300 | 24300 | <100 | <100 |
| | 9411 | 24300 | 24300 | 24300 | <100 | <100 |
| | 9412 | 24300 | 24300 | 24300 | <100 | <100 |
| | 9413 | >72900 | >72900 | >72900 | <100 | <100 |
| | 9414 | 72900 | 72900 | 72900 | <100 | <100 |
| | 9415 | >72900 | >72900 | 72900 | <100 | <100 |
| | 9416 | 24300 | 24300 | 24300 | <100 | <100 |
| | 9417 | 24300 | 24300 | 24300 | <100 | <100 |
| | 9418 | 24300 | 72900 | 24300 | <100 | <100 |
| | 8923 | 900 | 8100 | 2700 | 8100 | <100 |
| | 8924 | 72900 | 72900 | 72900 | <100 | <100 |
| | 8925 | 72900 | 72900 | 72900 | <100 | <100 |
| | 8926 | 24300 | 24300 | 24300 | <100 | <100 |
| | 9419 | 24300 | 24300 | 24300 | <100 | <100 |
| | 9420 | >72900 | >72900 | >72900 | <100 | <100 |

LHH-11 Tagged (HTL-CTL) HCV Protein (TR RDTX IM 57):

Mouse sera (07-019, group 1 till 6) were serially diluted on LHH-11 (IGP 3147) and LHH-11 -tagged (HTL-CTL)_HCV poly-epitope protein. Sera were tested in a ⅓ serial dilution starting from 1/100 to 1/72900.

Titers were calculated for each serial dilution as summarized in Table 7. For sera that showed antibody responses towards the LHH-11 peptide were compared to the antibody titers against the full length LHH-11 tagged (HTL-CTL)_HCV protein in Table 8.

TABLE 7
The antibody titers of the mouse sera tested in the peptide (IGP3147)
coating ELISA

| Group | Immunization scheme | Mouse N^{o} | Titer |
|---|---|---|---|
| 07-019/1 | 100 μg (HTL-CTL)-LHH11 | 12561 | <100 |
| | HCV protein (urea codition) | 12562 | <100 |
| | w0 and w2 | 12563 | <100 |
| | 100 μg (CTL-HTL)-HCV DNA | 12564 | <100 |
| | w5 | 12565 | <100 |
| | | 12566 | <100 |
| | | 12568 | <100 |
| | | 12569 | <100 |
| | | 12570 | <100 |
| | | 12571 | <100 |
| | | 12572 | <100 |
| | | 12573 | <100 |
| | | 12574 | <100 |
| | | 12575 | <100 |
| | | 12576 | <100 |
| | | 12577 | <100 |
| | | 12578 | <100 |
| 07-019/2 | 20 μg (HTL-CTL)-LHH11 | 12579 | <100 |
| | HCV protein (urea condition) | 12580 | <100 |
| | w0 and w2 | 12581 | <100 |
| | 100 μg (CTL-HTL)-HCV DNA | 12584 | <100 |
| | w5 | 12585 | <100 |
| | | 12586 | <100 |
| | | 12587 | <100 |
| | | 12588 | <100 |
| | | 12589 | <100 |
| | | 12590 | <100 |
| | | 12591 | <100 |
| | | 12592 | <100 |
| | | 12594 | <100 |
| | | 12595 | <100 |
| | | 12596 | <100 |
| 07-019/3 | 100 μg (HTL-CTL)-LHH11 | 12597 | <100 |
| | HCV protein_Alum | 12598 | <100 |
| | w0 and w2 | 12599 | <100 |
| | 100 μg (CTL-HTL)-HCV DNA | 12600 | <100 |
| | w5 | 12601 | <100 |
| | | 12602 | <100 |
| | | 12603 | <100 |
| | | 12604 | <100 |
| | | 12605 | 100 |
| | | 12606 | 100 |
| | | 12607 | <100 |
| | | 12608 | <100 |
| | | 12609 | <100 |
| | | 12610 | <100 |
| | | 12611 | <100 |
| | | 12612 | <100 |
| | | 12613 | <100 |
| | | 12614 | <100 |
| 07-019/4 | 20 μg (HTL-CTL)-LHH11 | 12615 | <100 |
| | HCV protein_Alum | 12616 | <100 |
| | w0 and w2 | 12617 | <100 |
| | 100 μg (CTL-HTL)-HCV DNA | 12618 | <100 |
| | w5 | 12619 | <100 |
| | | 12620 | <100 |
| | | 12621 | <100 |
| | | 12622 | <100 |
| | | 12623 | <100 |
| | | 12624 | <100 |
| | | 11653 | <100 |
| | | 12626 | <100 |
| | | 12627 | <100 |
| | | 12628 | <100 |
| | | 12629 | <100 |
| | | 12630 | <100 |
| | | 12631 | <100 |
| | | 12632 | <100 |
| 07-019/5 | 100 μg (HTL-CTL)-LHH11 | 12633 | <100 |
| | HCV protein (acetate condition) | 12634 | <100 |
| | w0 and w2 | 12635 | <100 |
| | 100 μg (CTL-HTL)-HCV DNA | 12636 | <100 |
| | w5 | 12637 | <100 |
| | | 12639 | <100 |
| | | 12640 | <100 |
| | | 12641 | <100 |
| | | 12643 | <100 |
| | | 12644 | <100 |
| | | 12645 | <100 |
| | | 12646 | <100 |
| | | 12647 | <100 |
| | | 12648 | <100 |
| | | 12649 | <100 |
| | | 12650 | <100 |
| 07-019/6 | 100 μg (HTL-CTL)-LHH11 | 12651 | <100 |
| | HCV protein aggregate | 12652 | <100 |
| | w0 and w2 | 12653 | <100 |
| | 100 μg (CTL-HTL)-HCV DNA | 12654 | <100 |
| | w5 | 12655 | <100 |
| | | 12656 | <100 |
| | | 12657 | <100 |
| | | 12658 | <100 |
| | | 12659 | <100 |
| | | 12660 | 100 |
| | | 12661 | 100 |
| | | 12662 | <100 |
| | | 12663 | 300 |
| | | 12664 | 100 |
| | | 12665 | <100 |
| | | 12666 | <100 |
| | | 12667 | <100 |
| | | 12668 | <100 |

TABLE 8
Comparison between the antibody titers against the
LHH-11 peptide and the full length LHH-11 tagged
(HTL-CTL) _HCV protein, for samples that showed
antibody responses towards the LHH-11 peptide

Titer against

| Mouse nr | LHH11 peptide | (HTL-CTL)-LHH11_HCV protein |
|---|---|---|
| 12605 | 100 | 6436 |
| 12606 | 100 | 14803 |
| 12660 | 100 | 72900 |
| 12661 | 100 | 900 |
| 12663 | 300 | 100 |
| 12664 | 100 | 8100 |

As only low reactivity against the LHH-11 tag was detected in 6 mice (titers of 100 to 300) and no correlation was seen with the positive antibody responses against the HCV poly-epitope protein, can be concluded that the LHH-tag in the poly-epitope protein does not induce a high humoral response.

### REFERENCES

Ausubel et al. (1992) Current Protocols in Molecular Biology, Greene/Wiley, New York, N.Y.

Ausubel, F. M., et al. Brent, R., Kingston, R. E., Moore, D. D., Seidman, J. G., Smith, J. A. & Struhl, K. Current Protocols in Molecular Biology; John Wiley and Sons, Inc., 2007

Gaberc-Porekar and Menart, J Biochem Biophys Methods 49 (2001), 335-360.

Gottesman S. et al. (1981) Cell, 24(1), 225-233

Harlow & Lane, Antibodies: A Laboratory Manual, Cold Spring Harbor Laboratory, Cold Spring Harbor, N.Y., 1988

Lichty J. J. et al., Protein Expression and Purification, 41, May 2005, Pages 98-105

Kim et al., Mol. Cells, 11(3), pp 297-294 (2001)

Kohler and Milstein, Nature, 256, pp. 495-497 (1975)

Metzger et al. (1988) Nature 334:31-36

Nakamura, Y., Gojobori, T. and Ikemura, T. (2000) Nucl. Acids Res. 28, 292.

Porath J. et al., Nature 258: 598-599, 1975

Sambrook et al. Molecular Cloning, a Laboratory Manual, 2nd Ed., Cold Spring Harbor, N.Y.:

Cold Spring Harbor Laboratory Press, pp. 16.30-16. 55 (1989)

Smith, M. C., Furman, T. C., Ingolia, T. D., Pidgeon, C. (1988) J. Biol. Chem. 263, 7211-7215

Watson, J. D., et al., Recombinant DNA, 2^{nd} Ed., New York: W. H. Freeman and Co. (1992)

Winnacker, E., From Genes to Clones, New York: VCH Publishers (1987)

Wu (ed) Molecular Cloning, Cold Spring Harbor Laboratory, Plainview, N.Y. (1993)

1. An affinity tag consisting of a sequence of 7 to 50 amino acids with the following characteristics:
comprising at least 6 Histidine residues whereby each His residue is followed by another His residue or by 1 to 4 non-His amino acids,
wherein at most 4 consecutive amino acids of the tag sequence are identical to a human protein amino acid sequence, and
wherein any window of 6 consecutive amino acids of the tag sequence can comprise up to 5 amino acids identical to a human protein sequence but with the provision that the remaining amino acid in the window is not similar to a human protein amino acid in said window.

2. An affinity tag according to claim 1, comprising the sequence:
(SEQ ID NO 36)
H(X₁)(X₂)(X₃)(X₄)H(X₅)(X₆)(X₇)(X₈)H(X₉)(X₁₀)(X₁₁)
(X₁₂)H(X₁₃)(X₁₄)(X₁₅)(X₁₆)H(X₁₇)(X₁₈)(X₁₉)(X₂₀)H,
wherein X₁_{_{—}}₂₀ are independently from each other either optional, or, if present selected from any non-His amino acid.

3. An affinity tag according to claim 2, wherein X₁₋₂₀ are independently from each other either optional, or, if present selected from the group of amino acids consisting of M, W, N and F.

4. An affinity tag according to claim 3, comprising the sequence:

| | |
|---|---|
| (H)HH-X₁-X₂-(H)HH, | (SEQ ID NO 37) |

wherein X₁-₂ are independently from each other selected from the group consisting of N, M, F and W.

5. An affinity tag according to claim 4, comprising the sequence
(SEQ ID NO 38)
(H)HH-X₁-X₂-(H)HH-X₃-X₄-(H)HH-X₅-X₆-(H)HH,
whereby X₁₋₆ are independently from each other selected from the group consisting of N, M, F and W.

6. The tag according to claim 1, further characterized in that the sequence comprises at least 8 Histidine (H) residues.

7. The tag according to claim 1, wherein said tag is a metal affinity tag.

8. A fusion molecule comprising the tag according to claim 1.

9. The fusion molecule according to claim 8, wherein the tag is coupled directly to the molecule or via a linker.

10. The fusion molecule according to claim 9, wherein the linker is a peptide sequence of 1 to 30 amino acids long.

11. The fusion molecule according to claim 8, wherein the molecule is a protein or a fragment thereof.

12. An isolated nucleic acid fragment coding for the affinity tag according to claim 1.

13. An isolated nucleic acid comprising a nucleic acid fragment according to claim 12.

14. An isolated nucleic acid coding for a fusion molecule according to claim 8.

15. A vector comprising a nucleic acid according to claim 12.

16. A host cell comprising the vector or nucleic acid according to claim 12.

17. A method of purification or immobilization of a molecule comprising use of the affinity tag according to claim 1, or the nucleic acid coding for same.

18. A method for purifying a fusion molecule comprising the steps of:
(a) applying a solution containing a fusion molecule according to claim 8 to a solid support possessing an immobilized affinity ligand,
(b) forming a complex between said immobilized affinity ligand and said molecule,
(c) removing weakly bounded molecules, and
(d) eluting the bound molecule.

19. The method according to claim 18 whereby the method further comprises a step (e) wherein the affinity tag is removed.

20. A composition comprising a fusion molecule according to claim 8.

21. A composition according to claim 20 which is a pharmaceutical composition.

22. A composition according to claim 21, further comprising at least one of a pharmaceutically acceptable excipient.

23. The fusion molecule according to claim 8, or the composition containing said fusion molecule for use as a medicament.

24. The fusion molecule or composition according to claim 23, wherein the molecule linked to the tag is an immunogenic compound.

25. A method for immobilizing a fusion molecule comprising the steps of:
(a) applying a solution containing a fusion molecule according to claim 8 to a solid support possessing an immobilized affinity ligand,
(b) forming a complex between said immobilized affinity ligand and said molecule, and
(c) removing weakly bounded molecules.

26. The method according to claim 18, wherein the affinity ligand is a metal ion charged IMAC ligand, an antibody, an antibody fragment, a small molecule or a synthetic affinity ligand.

27. A method for detecting a molecule in a sample by using the affinity tag according to claim 1, or the nucleic acid as a marker.

28. The affinity tag according to claim 1, wherein the tag comprises a sequence selected from the group consisting of the sequences represented by SEQ ID NO 1 to SEQ ID NO 35.

29. The affinity tag according to claim 28, wherein the tag comprises the sequence selected from the group consisting of: |HHHWWHHH (SEQ ID NO 1); HHH|
| | |
|---|---|
| HHHWWHHH; | (SEQ ID NO 1) |
| HHHWWHHH WWHHH; | (SEQ ID NO 17) |
| HHHWWHHHWWHHHWWHHH; | (SEQ ID NO 33) |
| HHMWHHHMWHHH; | (SEQ ID NO 13) |
| HHMWHHHMWHHHMWHHH; | (SEQ ID NO 31) |
| HHHMFHHNWHH; | (SEQ ID NO 12) |
| HHHMFHHHWWHHH; | (SEQ ID NO 22) |
| HHHWWHHHMWHHH; | (SEQ ID NO 23) |
| and | |
| HHHMFHHHWWHHHMWHHH. | (SEQ ID NO 32) |

30. A method for preparing the fusion molecule according to claim 8.

31. An antibody specifically binding the affinity tag or fusion molecule according to claim 1.


SEQUENCE LISTING (54 sequences)

SEQ ID NO: 1 - protein, 8 aa, Artificial Sequence
  Affinity tag
HHHWWHHH

SEQ ID NO: 2 - protein, 8 aa, Artificial Sequence
  Affinity tag
HHHMWHHH

SEQ ID NO: 3 - protein, 8 aa, Artificial Sequence
  Affinity tag
HHHWFHHH

SEQ ID NO: 4 - protein, 18 aa, Artificial Sequence
  Affinity tag
HHWWHHWWHHWWHHWWHH

SEQ ID NO: 5 - protein, 8 aa, Artificial Sequence
  Affinity tag
HHHNWHHH

SEQ ID NO: 6 - protein, 10 aa, Artificial Sequence
  Affinity tag
HHWWHHWWHH

SEQ ID NO: 7 - protein, 10 aa, Artificial Sequence
  Affinity tag
HHMMHHMMHH

SEQ ID NO: 8 - protein, 10 aa, Artificial Sequence
  Affinity tag
HHMFHHMFHH

SEQ ID NO: 9 - protein, 10 aa, Artificial Sequence
  Affinity tag
HHMWHHMWHH

SEQ ID NO: 10 - protein, 11 aa, Artificial Sequence
  Affinity tag
HWHWHWHWHWH

SEQ ID NO: 11 - protein, 13 aa, Artificial Sequence
  Affinity tag
HWHWHWHWHWHWH

SEQ ID NO: 12 - protein, 11 aa, Artificial Sequence
  Affinity tag
HHHMFHHNWHH

SEQ ID NO: 13 - protein, 12 aa, Artificial Sequence
  Affinity tag
HHMWHHHMWHHH

SEQ ID NO: 14 - protein, 14 aa, Artificial Sequence
  Affinity tag
HHMFHHMFHHMFHH

SEQ ID NO: 15 - protein, 12 aa, Artificial Sequence
  Affinity tag
HHMWHHHMFHHH

SEQ ID NO: 16 - protein, 13 aa, Artificial Sequence
  Affinity tag
HHHMWHHHMFHHH

SEQ ID NO: 17 - protein, 13 aa, Artificial Sequence
  Affinity tag
HHHWWHHHWWHHH

SEQ ID NO: 18 - protein, 13 aa, Artificial Sequence
  Affinity tag
HHHWFHHHWFHHH

SEQ ID NO: 19 - protein, 18 aa, Artificial Sequence
  Affinity tag
HHHMWHHHWWHHHMWHHH

SEQ ID NO: 20 - protein, 18 aa, Artificial Sequence
  Affinity tag
HHHWFHHHWFHHHWFHHH

SEQ ID NO: 21 - protein, 13 aa, Artificial Sequence
  Affinity tag
HHHMWHHHWWHHH

SEQ ID NO: 22 - protein, 13 aa, Artificial Sequence
  Affinity tag
HHHMFHHHWWHHH

SEQ ID NO: 23 - protein, 13 aa, Artificial Sequence
  Affinity tag
HHHWWHHHMWHHH

SEQ ID NO: 24 - protein, 14 aa, Artificial Sequence
  Affinity tag
HHWWHHWWHHWWHH

SEQ ID NO: 25 - protein, 14 aa, Artificial Sequence
  Affinity tag
HHMMHHMMHHMMHH

SEQ ID NO: 26 - protein, 14 aa, Artificial Sequence
  Affinity tag
HHMWHHMWHHMWHH

SEQ ID NO: 27 - protein, 13 aa, Artificial Sequence
  Affinity tag
HHHMWHHHMWHHH

SEQ ID NO: 28 - protein, 17 aa, Artificial Sequence
  Affinity tag
HHMWHHHMFHHHWWHHH

SEQ ID NO: 29 - protein, 18 aa, Artificial Sequence
  Affinity tag
HHHMWHHHMFHHHWWHHH

SEQ ID NO: 30 - protein, 18 aa, Artificial Sequence
  Affinity tag
HHMWHHMWHHMWHHMWHH

SEQ ID NO: 31 - protein, 17 aa, Artificial Sequence
  Affinity tag
HHMWHHHMWHHHMWHHH

SEQ ID NO: 32 - protein, 18 aa, Artificial Sequence
  Affinity tag
HHHMFHHHWWHHHMWHHH

SEQ ID NO: 33 - protein, 18 aa, Artificial Sequence
  Affinity tag
HHHWWHHHWWHHHWWHHH

SEQ ID NO: 34 - protein, 18 aa, Artificial Sequence
  Affinity tag
HHHMWHHHMWHHHMWHHH

SEQ ID NO: 35 - protein, 18 aa, Artificial Sequence
  Affinity tag
HHHWWHHHMWHHHWWHHH

SEQ ID NO: 36 - protein, 26 aa, Artificial Sequence
  Affinity tag
HXXXXHXXXXHXXXXHXXXXHXXXXH

SEQ ID NO: 37 - protein, 8 aa, Artificial Sequence
  Affinity tag
HHHWWHHH

SEQ ID NO: 38 - protein, 18 aa, Artificial Sequence
  Affinity tag
HHHWWHHHWWHHHWWHHH

SEQ ID NO: 39 - protein, 6 aa, Artificial Sequence
  Linker sequence
EEGEPK

SEQ ID NO: 40 - protein, 6 aa, Artificial Sequence
  Linker sequence
EEAEPK

SEQ ID NO: 41 - protein, 4 aa, Artificial Sequence
  Linker sequence
IEGR

SEQ ID NO: 42 - protein, 4 aa, Artificial Sequence
  Linker sequence
LVPR

SEQ ID NO: 43 - protein, 5 aa, Artificial Sequence
  Linker sequence
DDDDK

SEQ ID NO: 44 - protein, 725 aa, Artificial Sequence
  HBV Polyepitope protein
MGTSFVYVPSALNPADGPGPGLCQVFADATPTGWGLGPGPGRHYLHTLWKAGILYKGPGPGPHHTALRQAILCWGELMTLAGPGPGESRLVVDFSQFSRGNGPGPGPFLLAQFTSAICSVVGPGPGLVPFVQWFVGLSPTVGPGPGLHLYSHPIILGFRKIGPGPGSSNLSWLSLDVSAAFGPGPGLQSLTNLLSSNLSWLGPGPGAGFFLLTRILTIPQSGPGPGVSFGVWIRTPPAYRPPNAPIGPGPGVGPLTVNEKRRLKLIGPGPGKQCFRKLPVNRPIDWGPGPGAANWILRGTSFVYVPGPGPGKQAFTFSPTYKAFLCGPGPGFLLSLGIHLNAAAKYTSFPWLLNAAARFSWLSLLVPFNAAFPHCLAFSYMKAALVVDFSQFSRGAILLLCLIFLLNAAAHTLWKAGILYKKAWMMWYWGPSLYKAYPALMPLYACIGAAAWLSLLVPFVNAAAGFLLTRILTINAAAIPIPSSWAFKAAAEYLVSFGVWNLPSDFFPSVKAAAFLPSDFFPSVKAAADLLDTASALYNSWPKFAVPNLKAAASAICSVVRRKLSLDVSAAFYNAAAKFVAAWTLKAAAKAANVSIPWTHKGAAGLSRYVARLNAAASTLPETTVVRRKHPAAMPHLLKAAARWMCLRRFIINASFCGSPYKAAYMDDVVLGVNALWFHISCLTFKAAATPARVTGGVFKAAALTFGRETVLEYKQAFTFSPTYK

SEQ ID NO: 45 - protein, 665 aa, Artificial Sequence
  HCV Polyepitope protein
MGRHLIFCHSKKKCDGPGPGSTLLFNILGGWVAAQGPGPGCVTQTVDFSLDPTFTIETTGPGPGEDLVNLLPAILSPGALVVGPGPGGEGAVQWMNRLIAFASGPGPGMNRLIAFASRGNHVSPGPGPGSMSYTWTGALITPCAGPGPGTPAETTVRLRAYMNTPGLPVGPGPGAVGIFRAAVCTRGVAGPGPGGIQYLAGLSTLPGNPAGPGPGTSTWVLVGGVLAALAAGPGPGGYKVLVLNPSVAATGPGPGGKPAIIPDREVLYREKAVIKGGRHLIKAGPRLGVRATKAAAQYLAGLSTLNAAAPTLWARMILNAAHPNIEEVALNLVDILAGYGAKHMWNFISGINAYYRGLDVSVKLQDCTMLVNAAAAEQFKQKALKTSERSQPRNAAFPYLVAYQAKAAMYTNVDQDLNTLWARMILMNLPINALSNSLKSTNPKPQRKNDYPYRLWHYKAACLIRLKPTLNIIMYAPTLKAAVATDALMTGYNLPGCSFSIFKYRRCRASGVLKAAVLVGGVLAALNGLLGCIITSLNAAYAAQGYKKDPRRRSRNLKAAAYLLPRRGPRLNFWAKHMWNFIKAAAKFVAAWTLKAAAKALIRLKPTLHKAAAVCTRGVAKNFTDNSSPPAVKALPRRGPRLGVKSFSIFLLALKAAETAGARLV

SEQ ID NO: 46 - DNA, 2208 bp, Artificial Sequence
  Fusion construct encoding HBV polyepitope protein linked to an affinity tag
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgcatcaccattggtggcaccatcattaa

SEQ ID NO: 47 - DNA, 2235 bp, Artificial Sequence
  Fusion construct encoding HBV polyepitope protein linked to an affinity tag
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgcatcacatgtggcaccatcatatgtggcatcatcacatgtggcaccatcactaa

SEQ ID NO: 48 - DNA, 2217 bp, Artificial Sequence
  Fusion construct encoding HBV polyepitope protein linked to an affinity tag
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgcatcaccatatgtttcaccataactggcatcactaa

SEQ ID NO: 49 - DNA, 2223 bp, Artificial Sequence
  Fusion construct encoding HBV polyepitope protein linked to an affinity tag
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgcatcaccatatgtttcaccatcattggtggcatcatcactaa

SEQ ID NO: 50 - DNA, 2238 bp, Artificial Sequence
  Fusion construct encoding HBV polyepitope protein linked to an affinity tag
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgcatcaccatatgtttcaccatcattggtggcatcatcacatgtggcaccatcactaa

SEQ ID NO: 51 - DNA, 2040 bp, Artificial Sequence
  Fusion construct encoding HCV polyepitope protein linked to an affinity tag
atgggccgtcatctgattttttgccacagcaaaaaaaaatgcgatgggccgggtcctggtagcaccctgctgtttaacattctgggcggctgggttgcggcgcagggtccaggtccgggctgtgttacccagaccgtggattttagcctggacccgacctttaccattgaaaccactgggccaggtcctggggaggacctggtgaacctgctgccggcgattctgtctccgggtgcgctggttgttggtccaggtccggggggtgaaggtgcggtgcagtggatgaaccgtctgattgcgtttgcgtctggtccagggccaggtatgaaccgcctgatcgcgttcgccagccgtggcaaccatgtttctccgggtccagggccaggtagcatgagctatacctggaccggtgcgctgattaccccgtgtgcaggtccagggccgggtacgccggcggaaaccaccgttcgtctgcgtgcgtatatgaacacgccgggtctgccggtgggtccaggtccaggtgcggtgggcatttttcgtgcggcggtgtgcacccgtggtgttgcaggcccaggtccagggggtattcagtatctggccggcctgagcaccctgccgggtaatccggcgggtccgggtccaggcacctctacctgggtgctggttggtggtgttctggccgcgctggccgcaggtccgggtccagggggctataaagtgctggtgctgaatccgagcgttgcggcgaccggtccgggtccgggtggtaaaccggcgattattccggatcgtgaagtgctgtatcgtgaaaaagcggtgattaaaggcggccgtcatctgattaaagcgggtccacgcctgggtgttcgtgcgaccaaagccgcagcgcagtatctggccggtctgagcaccctgaatgcggcagcgccgacgctgtgggcgcgtatgattctgaacgcggcgcatccgaacattgaagaagtggcgctgaacctggtggatattctggccggctatggcgcaaaacatatgtggaactttatcagcggcatcaacgcgtattatcgtggcctggatgtgagcgtgaaactgcaggattgcaccatgctggtgaatgcggccgcagcggaacagtttaaacagaaagcgctgaaaaccagcgaacgtagccagccgcgtaacgcggcgtttccgtatctggtggcgtatcaggcgaaagcggcgatgtataccaacgtggatcaggatctgaacaccctgtgggcccgcatgatcctgatgaacctgccgattaacgccctgagcaacagcctgaaaagcaccaatccgaaaccgcagcgtaaaaacgattatccgtatcgcctgtggcattataaagcggcgtgcctgattcgtctgaaaccaactctgaacattattatgtatgccccgacgctgaaagcagccgttgcgaccgatgcgctgatgaccggctataacctgccgggctgcagctttagcatttttaaatatcgtcgttgccgtgcgagcggcgttctgaaagctgcggtgctggttggtggtgttctggccgcgctgaacggtctgctgggctgcattattaccagcctgaacgcggcctatgcggcgcagggctataaaaaagatccgcgtcgtcgtagccgtaacctgaaagcagcggcgtatctgctgccgcgccgtggcccgcgtctgaacttttgggcgaaacacatgtggaatttcattaaagccgcggccaaatttgtggcggcgtggaccctgaaagccgccgcgaaagcgctgatccgcctgaaaccgaccctgcataaagcggcagcggtgtgcacccgtggcgtggcgaaaaactttaccgataacagctctccgccggcagttaaagccctgccgcgtcgcggtccgcgcctgggcgtgaaatcttttagcatctttctgctggccctgaaagcggcggaaaccgcgggtgcgcgtctggttaatgcggcgcatcatcatatgtttcatcacaactggcatcattaa

SEQ ID NO: 52 - DNA, 2061 bp, Artificial Sequence
  Fusion construct encoding HCV polyepitope protein linked to an affinity tag
atgggccgtcatctgattttttgccacagcaaaaaaaaatgcgatgggccgggtcctggtagcaccctgctgtttaacattctgggcggctgggttgcggcgcagggtccaggtccgggctgtgttacccagaccgtggattttagcctggacccgacctttaccattgaaaccactgggccaggtcctggggaggacctggtgaacctgctgccggcgattctgtctccgggtgcgctggttgttggtccaggtccggggggtgaaggtgcggtgcagtggatgaaccgtctgattgcgtttgcgtctggtccagggccaggtatgaaccgcctgatcgcgttcgccagccgtggcaaccatgtttctccgggtccagggccaggtagcatgagctatacctggaccggtgcgctgattaccccgtgtgcaggtccagggccgggtacgccggcggaaaccaccgttcgtctgcgtgcgtatatgaacacgccgggtctgccggtgggtccaggtccaggtgcggtgggcatttttcgtgcggcggtgtgcacccgtggtgttgcaggcccaggtccagggggtattcagtatctggccggcctgagcaccctgccgggtaatccggcgggtccgggtccaggcacctctacctgggtgctggttggtggtgttctggccgcgctggccgcaggtccgggtccagggggctataaagtgctggtgctgaatccgagcgttgcggcgaccggtccgggtccgggtggtaaaccggcgattattccggatcgtgaagtgctgtatcgtgaaaaagcggtgattaaaggcggccgtcatctgattaaagcgggtccacgcctgggtgttcgtgcgaccaaagccgcagcgcagtatctggccggtctgagcaccctgaatgcggcagcgccgacgctgtgggcgcgtatgattctgaacgcggcgcatccgaacattgaagaagtggcgctgaacctggtggatattctggccggctatggcgcaaaacatatgtggaactttatcagcggcatcaacgcgtattatcgtggcctggatgtgagcgtgaaactgcaggattgcaccatgctggtgaatgcggccgcagcggaacagtttaaacagaaagcgctgaaaaccagcgaacgtagccagccgcgtaacgcggcgtttccgtatctggtggcgtatcaggcgaaagcggcgatgtataccaacgtggatcaggatctgaacaccctgtgggcccgcatgatcctgatgaacctgccgattaacgccctgagcaacagcctgaaaagcaccaatccgaaaccgcagcgtaaaaacgattatccgtatcgcctgtggcattataaagcggcgtgcctgattcgtctgaaaccaactctgaacattattatgtatgccccgacgctgaaagcagccgttgcgaccgatgcgctgatgaccggctataacctgccgggctgcagctttagcatttttaaatatcgtcgttgccgtgcgagcggcgttctgaaagctgcggtgctggttggtggtgttctggccgcgctgaacggtctgctgggctgcattattaccagcctgaacgcggcctatgcggcgcagggctataaaaaagatccgcgtcgtcgtagccgtaacctgaaagcagcggcgtatctgctgccgcgccgtggcccgcgtctgaacttttgggcgaaacacatgtggaatttcattaaagccgcggccaaatttgtggcggcgtggaccctgaaagccgccgcgaaagcgctgatccgcctgaaaccgaccctgcataaagcggcagcggtgtgcacccgtggcgtggcgaaaaactttaccgataacagctctccgccggcagttaaagccctgccgcgtcgcggtccgcgcctgggcgtgaaatcttttagcatctttctgctggccctgaaagcggcggaaaccgcgggtgcgcgtctggttaatgcggcgcatcatcatatgtttcaccatcattggtggcatcatcacatgtggcaccatcactaa

SEQ ID NO: 53 - DNA, 4947 bp, Artificial Sequence
  pAcI plasmid
gttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcaggtgatgattatcagccagcagagaattaaggaaaacagacaggtttattgagcgcttatctttccctttatttttgctgcggtaagtcgcataaaaaccattcttcataattcaatccatttactatgttatgttctgaggggagtgaaaattcccctaattcgatgaagattcttgctcaattgttatcagctatgcgccgaccagaacaccttgccgatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgtaccgagctcgaattgctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgt

SEQ ID NO: 54 - DNA, 4182 bp, Artificial Sequence
  pcI857 plasmid
tctcgatgatggttacgccagactatcaaatatgctgcttgaggcttattcgggcgcagatctttagctgtcttggtttgcccaaagcgcattgcataatctttcagggttatgcgttgttccatacaacctccttagtacatgcaaccattatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggagga